SEQUENCE LISTING (18 sequences)

SEQ ID NO: 1 - protein, 293 aa, Thermus thermophilus
MRPIEHALSYAAQGYGVLPLRPGGKEPLGKLVPHGLKNASRDPATLEAWWRSCPRCGVGILPGPEVLVLDFDDPEAWEGLRQEHPALEAAPRQRTPKGGRHVFLRLPEGVRLSASVRAIPGVDLRGMGRAYVVAAPTRLKDGRTYTWEAPLTPPEELPPVPQALLLKLLPPPPPPRPSWGAVGTASPKRLQALLQAYAAQVARTPEGQRHLTLIRYAVAAGGLIPHGLDPREAEEVLVAAAMSAGLPEWEARDAVRWGLGVGASRPLVLESSSKPPEPRTYRARVYARMRRWV

SEQ ID NO: 2 - protein, 631 aa, Artificial
  TelN
MSKVKIGELINTLVNEVEAIDASDRPQGDKTKRIKAAAARYKNALFNDKRKFRGKGLQKRITANTFNAYMSRARKRFDDKLHHSFDKNINKLSEKYPLYSEELSSWLSMPTANIRQHMSSLQSKLKEIMPLAEELSNVRIGSKGSDAKIARLIKKYPDWSFALSDLNSDDWKERRDYLYKLFQQGSALLEELHQLKVNHEVLYHLQLSPAERTSIQQRWADVLREKKRNVVVIDYPTYMQSIYDILNNPATLFSLNTRSGMAPLAFALAAVSGRRMIEIMFQGEFAVSGKYTVNFSGQAKKRSEDKSVTRTIYTLCEAKLFVELLTELRSCSAASDFDEVVKGYGKDDTRSENGRINAILAKAFNPWVKSFFGDDRRVYKDSRAIYARIAYEMFFRVDPRWKNVDEDVFFMEILGHDDENTQLHYKQFKLANFSRTWRPEVGDENTRLVALQKLDDEMPGFARGDAGVRLHETVKQLVEQDPSAKITNSTLRAFKFSPTMISRYLEFAADALGQFVGENGQWQLKIETPAIVLPDEESVETIDEPDDESQDDELDEDEIELDEGGGDEPTEEEGPEEHQPTALKPVFKPAKNNGDGTYKIEFEYDGKHYAWSGPADSPMAAMRSAWETYYS

SEQ ID NO: 3 - DNA, 56 bp, Artificial
  TelN target sequence
tatcagcacacaattgcccattatacgcgcgtataatggactattgtgtgctgata

SEQ ID NO: 4 - DNA, 141 bp, Artificial
  5′ ITR sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 141 bp, Artificial
  3′ ITR sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 17 bp, Artificial Sequence
  oligo 15
agggatccactcaggat

SEQ ID NO: 7 - DNA, 17 bp, Artificial Sequence
  oligo 37
  modified_base (9)..(13): phosphothioated nucleotides
agggatccactcaggat

SEQ ID NO: 8 - DNA, 21 bp, Artificial Sequence
  oligo 4
agggctaaccactcaggttag

SEQ ID NO: 9 - DNA, 21 bp, Artificial Sequence
  oligo 28
  modified_base (11)..(11): 8-oxo-deoxyadenosine
  modified_base (15)..(15): 8-oxo-deoxyadenosine
agggctaaccactcaggttag

SEQ ID NO: 10 - DNA, 21 bp, Artificial Sequence
  oligo 29
  modified_base (12)..(12): 5-Fluoro-deoxyuracil
  misc_feature (12)..(12): n is a, c, g, or t
  modified_base (14)..(14): 5-Fluoro-deoxyuracil
  misc_feature (14)..(14): n is a, c, g, or t
agggctaaccantnaggttag

SEQ ID NO: 11 - DNA, 31 bp, Artificial Sequence
  oligo 17
agggataacatggccactcaggccatgttat

SEQ ID NO: 12 - DNA, 25 bp, Artificial Sequence
  oligo 19
  modified_base (11)..(15): LNA nucleotides
agggataacatggccactcaggcca

SEQ ID NO: 13 - DNA, 31 bp, Artificial Sequence; identical to SEQ ID NO: 11
  oligo 22
  modified_base (16)..(16): 8-oxo-deoxyadenosine
  modified_base (20)..(20): 8-oxo-deoxyadenosine

SEQ ID NO: 14 - DNA, 31 bp, Artificial Sequence
  oligo 21
  modified_base (16)..(16): I
  misc_feature (16)..(16): n is a, c, g, or t
  modified_base (20)..(20): I
  misc_feature (20)..(20): n is a, c, g, or t
agggataacatggccnctcnggccatgttat

SEQ ID NO: 15 - DNA, 18 bp, Artificial Sequence
  oligo 41
  modified_base (11)..(14): phosphothioated nucleotide
agggcttacgcgcgtaag

SEQ ID NO: 16 - DNA, 3762 bp, Artificial Sequence
  eGFP plasmid
ccaatgatggtaccgtatcagcacacaattgcccattatacgcgcgtataatggactattgtgtgctgataggtctcgccctgatttaaatcgtcgaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatttaaatcggtctcgagggtatcagcacacaattgcccattatacgcgcgtataatggactattgtgtgctgatagaagcttatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 17 - DNA, 4695 bp, Artificial Sequence
  Luc plasmid
ccaatgatggtaccgtatcagcacacaattgcccattatacgcgcgtataatggactattgtgtgctgataggtctcgccctgatttaaatcgtcgaccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcccgggatccaccggtcgccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatttaaatcggtctcgagggtatcagcacacaattgcccattatacgcgcgtataatggactattgtgtgctgatagaagcttatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 18 - DNA, 4902 bp, Artificial Sequence
  Luc-ITR plasmid
ccaatgatggtaccgtatcagcacacaattgcccattatacgcgcgtataatggactattgtgtgctgatagataggtctcgccctcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgctagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtcaagccatggcttcccgccggcggtggcggcgcaggatgatggcacgctgcccatgtcttgtgcccaggagagcgggatggaccgtcaccctgcagcctgtgcttctgctaggatcaatgtgtaggcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggtctcgagggtatcagcacacaattgcccattatacgcgcgtataatggactattgtgtgctgatagaagcttatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2023/0323343 A1
(43) Pub. Date: Oct. 12, 2023

(54) CLOSED LINEAR DNA WITH MODIFIED NUCLEOTIDES

(71) Applicant: TYRIS THERAPEUTICS, S.L., Moncada (ES)

(72) Inventor: Julen OYARZABAL SANTAMARINA, MADRID (ES)

(21) Appl. No.: 17/796,532

(22) PCT Filed: Jan. 29, 2021
(86) PCT No.: PCT/EP2021/052204

§ 371 Date: Jul. 29, 2022

(30) Foreign Application Priority Data
Jan. 31, 2020 (EP) 20382063.4

(51) Int. Cl. C12N 15/113

(52) U.S. Cl. CPC C12N 15/113; C12N 2310/531; C12N 2310/314; C12N 2310/3231

(57) ABSTRACT

The present invention provides closed linear DNA (clDNA) consisting of a stem region comprising a double stranded DNA sequence of interest covalently closed at both ends by hairpin loops, the clDNA comprising at least two modified nucleotides. The invention also provides the clDNA for use in therapy, in particular, gene therapy, as well as pharmaceutical compositions comprising the clDNA and a method for the production of the clDNA.

This application claims the benefit of European Patent Application EP20382063.4 filed on 31 Jan. 2020.

### TECHNICAL FIELD

The present invention belongs to the field of nucleic acids. In particular, the invention relates to closed linear DNA that contain modified nucleotides. The closed linear DNA of the present invention is particular useful for therapeutic purposes.

### BACKGROUND ART

Gene therapy holds great promise for the treatment of several disease. It is based on the successful transfer of genetic material into the nuclei of targeted human cells. Gene delivery systems can be viral or non-viral in design. Compared with viral DNA vectors, non-viral transgene delivery systems offer safer gene transfer and vaccine design approaches, are less likely to elicit inflammatory and immune responses in hosts, have greater transgene capacity, and are easier to store.

However, the effectiveness of non-viral vectors is very limited, which has hindered their introduction to the clinic. For instance, the use of conventional plasmid DNA vectors for gene therapy can elicit adverse immune responses due to bacterial sequences they contain, and their bioavailability is compromised because of their large molecular size. Therefore, new types of non-viral DNA constructs have been developed in recent years.

In this regard, the use of small linear oligodeoxynucleotides (ODN) that only carry the DNA sequence of interest—without the bulk of an immunogenic bacterial backbone—has been extensively explored. However, ODNs are prone to degradation by endonucleases and exonucleases which has greatly limited their therapeutic potential.

In order to improve ODN stability, several strategies have been followed in the prior art. On the one hand, open linear ODNs have been chemically modified to ensure their persistence in vivo. For instance, L-DNA nucleotides have been included at their open ends to protect them against nucleolytic degradation (Kapp K et al., “EnanDIM—a novel family of L-nucleotide-protected TLR9 agonists for cancer immunotherapy” 2019, J Immunother Cancer., vol 7(1), pp. 5). However, the experimental results have been modest so far and the addition of modified nucleotides within these open DNA structures often leads to off-target side effects.

Another strategy for ODN stabilization has consisted on the formation of closed linear DNA (clDNA) molecules wherein the double stranded region is flanked and protected by two single stranded loops thereby generating dumbbell-shaped molecules. The absence of any open end in the clDNAs makes them highly resistant to nucleolytic degradation (Heinrich J. et al., “Linear closed mini DNA generated by the prokaryotic cleaving-joining enzyme TeIN is functional in mammalian cells” 2002, J Mol Med, vol. 80(10), pp. 648-54).

There have also been attempts to further improve clDNA stability by modifying the stem length and the loop size, or by incorporating sequence motives within the stem-loop regions. For instance, the presence of cytosine-guanine pairs at the closing of the loop has been related to increase loop stability. However, CG motives are known to be very potent immunostimulating sequences which greatly hinder their use in vectors directed to gene therapy, where the activation of the immune system is to be avoided.

Thus, a need remains for stable clDNAs that are suitable for in vivo expressing any given gene of interest without causing unwanted immune-related side effects.

### SUMMARY OF INVENTION

The present inventors have developed novel closed linear DNA (clDNA) which is suitable for use in DNA-based therapies, such as gene therapy. In particular, the clDNA of the invention includes at least two modified nucleotides which, together with the closed structure of the molecule, improves efficiency of the clDNA when used in DNA-based therapy.

Surprisingly, the inventor found that stable clDNAs could be produced even when they incorporated two or more nucleotide modifications. This was highly unexpected because clDNAs are very small molecules whose stability and functionality are highly dependent on their particular dumbbell-like shape. The prior art shows that most attempts to increase clDNA stability have been based on small modifications of the nucleotide sequence identity or at most on the addition of one single nucleotide modification in order not to disturb the fragile intramolecular interactions that maintain the clDNA structure.

The clDNAs of the invention constitute a very useful alternative to the constructions disclosed in the prior art for treating disease by DNA-based therapies, such as gene therapy.

Thus, in a first aspect, the invention provides a closed linear DNA (“clDNA”) consisting of a stem region comprising a double stranded DNA sequence of interest covalently closed at both ends by hairpin loops, the clDNA comprising at least two modified nucleotides. Advantageously, the at least two modified nucleotides may be incorporated in various regions of the molecule, such as the single stranded loop or particular regions of the stem, in order to modulate the characteristics of the clDNA to be synthesized.

The clDNAs of the invention comprising at least two modified oligonucleotides have several convenient properties that renders them advantageous with respect to their natural counterparts, such as increased transfection efficiency, expression efficiency, better stability, bioavailability, functional persistence, resistance to degradation and overall functional performance of the sequence of interest contained therein. The examples below demonstrate that several clDNAs containing modified nucleotides provide for a surprising improvement in the functional performance of the sequence of interest, which, in this case, and for the sake of providing a proof of concept, was luciferase activity.

The clDNAs of the invention are useful for multiple indications, for example, for therapeutic or diagnostic indications. In a second aspect, the invention provides the closed linear DNA according to the first aspect for use in therapy. In another aspect the invention provides the clDNA according to the first aspect of the invention for use in diagnosis.

In a third aspect, the invention provides a pharmaceutical composition comprising a therapeutically effective amount of the closed linear DNA according to the first aspect and pharmaceutically acceptable carriers or excipients.

In a fourth aspect, the invention provides process for the production of a closed linear DNA comprising at least two modified nucleotides according to this first aspect, comprising the steps of a) providing a DNA template comprising a DNA sequence of interest; b) amplifying DNA from the DNA template of step (a) producing a concatameric DNA comprising repeats of the DNA sequence of interest, wherein each one of the repeated DNA sequences of interest is flanked by restriction sites; c) generating a closed linear DNA with the amplified DNA produced in step (b) by (c.1) contacting the concatameric DNA with at least one restriction enzyme thereby producing a plurality of open double stranded DNA fragments each containing the DNA sequence of interest, and (c.2) attaching a hairpin DNA adaptor at each one of the ends of the open double stranded DNA fragments, wherein each one of the adaptors has at least one modified nucleotide or, alternatively, only one of the adaptors attached to the DNA fragment comprises the at least two modified nucleotide, and d) purifying the closed linear DNA produced in step (c).

In a fifth aspect, the invention provides a closed linear DNA obtainable by the process according to the fourth aspect. The invention also provides for the clDNA according to the fourth aspect for use in therapy or diagnosis.

In a sixth aspect, the invention provides a kit for the production of clDNA comprising hairpin DNA adaptors containing at least one modified nucleotide, a ligase, and optionally, instructions for its use.

The clDNAs may be provided on their own or together with a gene vector or carrier, or together with other DNA molecules which contribute to the desired therapeutic effect. The combination of the clDNA and a viral or non-viral vector, nanoparticle, or any other carrier may be convenient, for example, in order to target the desired cells or tissues. Indeed, the complexes formed by certain non-viral vectors and the clDNA containing modified nucleotides (herein also called polyplexes) may further improve certain properties such as transfection efficiency of the clDNAs to the desired cells or the release profile of the clDNA in physiological conditions. Non-limited non-viral vectors which are appropriate for forming a polyplex with the clDNAs of the invention are polycationic polymers.

Thus, in a seventh aspect, the invention provides a composition comprising a clDNA as defined in the first or fourth aspects of the invention and a carrier.

In an eight aspect, the invention provides a polyplex comprising a polymer, for example, a polycationic polymer, and a clDNA as defined in the first or fourth aspects of the invention.

### BRIEF DESCRIPTION OF DRAWINGS

FIG. 1 shows (A) the structure of a closed linear DNA according to the invention which consists of two stem-loop adapters flanking a DNA sequence of interest. (B) shows in more detail the structure of the adaptors forming the clDNA of the invention, wherein the stem of the adaptors presents a proximal region (1) at the end of the stem to be linked to the DNA sequence of interest, and a distal region (2) at the end of the stem that is closed by the single stranded loop.

FIG. 2. Shows preparation scheme for clDNAs prepared with customized hairpin adaptors. The DNA fragment comprising the sequence of interest (e.g. luciferase or Gfp) flanked at each side by endonuclease restriction sites (e.g. Bsal restriction sites) (A), was treated with the specific restriction endonuclease (B) and ligated with the desired hairpin adaptors (e.g. oligo 37 with SEQ ID NO: 7, which contains 5 phosphothioated nucleotides, shown in italics) and exonuclease to yield clDNA comprising modified nucleotides (C).

FIG. 3 shows quality control parameters for oDNA 17. A, Agarose gel electrophoresis (M1, supercoiled DNA Ladder Marker TAKARA: 3585A; M2, 1 kb DNA Ladder TIAGEN MD111; lane 11, oDNA 17); B, Grayscale analysis; C, anion-exchange chromatography-HPLC; D, Sanger Sequencing.

FIG. 4 shows quality control parameters for oDNA 19. A, Agarose gel electrophoresis (M1, supercoiled DNA Ladder Marker TAKARA: 3585A; M2, 1 kb DNA Ladder TIAGEN MD111; lane 2, oDNA 19); B, Grayscale analysis; C, anion-exchange chromatography-HPLC; D, Sanger Sequencing.

FIG. 5 shows quality control parameters for oDNA 41. A, Agarose gel electrophoresis (M1, supercoiled DNA Ladder Marker TAKARA: 3585A; M2, 1 kb DNA Ladder TIAGEN MD111; lane 5, oDNA 41); B, Grayscale analysis; D, Sanger Sequencing.

FIG. 6 shows luciferase activity on HaCaT cells transfected with clDNAs comprising natural (oDNA 15, oDNA 4 or oDNA 17) or modified (oDNA 37, oDNA 28, oDNA 19 or oDNA 22) oligonucleotides using PEI. A, 24 hours after transfection. B, 48 hours after transfection. *p<0.05; **p<0.01; ***p<0,001; ****p<0,0001 natural vs. its corresponding modified oDNAs (15 vs. 37, 4 vs. 28 and 17 vs. 19 or 22). Student t-test (n=3).

FIG. 7 shows the evolution of luciferase activity level vs time for HaCaT cells transfected with clDNAs comprising natural or modified oligonucleotides using PEI. Pairwise comparisons, natural vs. modified: A, oDNA 15 vs. oDNA 37; B, oDNA 4 vs. oDNA 28; and, C, oDNA 17 vs. oDNA 19 or oDNA 22. *p<0.05; **p<0.01; ***p<0,001; ****p<0,0001 the value of day 2 vs. the value of day_1. Student t-test (n=3).

FIG. 8 shows the release of clDNA cargo after 12 hours incubation, using Heparin at 8 U/mL which recapitulate physiological conditions releasing the complexed clDNA (competition for the polymer), from polyplexes formed by the polymer CXP-37 and clDNAs comprising natural (oDNA 15, oDNA 4) or modified (oDNA 37, oDNA 28, oDNA 29) oligonucleotides. *p<0.05; **p<0.01; ***p<0,001; ****p<0,0001 natural vs. its corresponding modified oDNAs (15 vs. 37, 4 vs. 28 or 29). Student t-test (n=3).

FIG. 9. Shows: A, Synthetic route of PAspDET/DIIPA. B, Synthesis of poly(β-benzyl L-aspartate) (PBLA). B, Synthesis of PAsp(DET/DIIPA)-Compound CXP037A.

FIG. 10 shows: 1H NMR spectrum of PBLA. ¹H NMR (DMSO-d₆): δ=0.79 (t, J=7.58 Hz, 3H), 1.13-1.37 (m, 4H), 2.96-2.52 (m, 2H, CH₂), 4.61 (s, 1H, CH), 5.01 (s, 2H, benzyl CH₂), 7.27 (s, 5H, aryl CH), 8.15 (s, 1H, NH).

FIG. 11 shows 1H NMR spectrum of CXP037. ¹H NMR (D2O): δ=0.84 (t, J=7.68 Hz, 3H), 1.32 (m, 3H, CH₃), 2.82 (brs, 2H, CH₂), 3.08-3.79 (m, 2H, CH₂).

FIG. 12 shows SEC-MALS-RI of CXP037A Analysis for MW determination. MW=14000 Da (1.03).

FIG. 13 shows Potentiometric titration curve for pKa determination of CXP037. Calculated pKA:5,370/8,952.

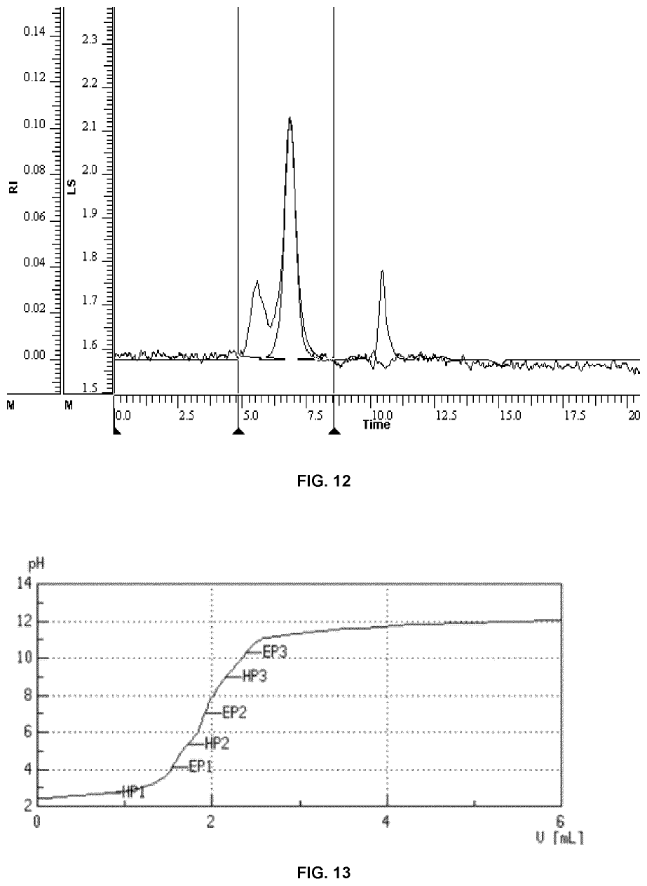

FIG. 14 shows representation of a fragment of eGFP plasmid (the plasmid having SEQ ID NO: 16) containing the sequence of interest for preparation of clDNA of the invention. The represented fragment comprises the sequence of interest (in this case the sequence encoding for GFP) together with additional sequences such as corresponding promoter and enhancer. The sequence of interest is flanked by Bsal restriction sites and protelomerase target sequences

FIG. 15 shows representation of a fragment of Luc-ITR (the plasmid having SEQ ID NO: 18) containing the sequence of interest for preparation of clDNA of the invention. The represented fragment comprises the sequence of interest (in this case the sequence encoding for Luciferase) together with additional sequences such as corresponding promoter and enhancer, as well as AVV2-ITRs. The sequence of interest is flanked by Bsal restriction sites and protelomerase target sequences.

FIG. 16 shows Agarose gel electrophoresis of oDNA 37_{ITR} (M, DL3000 ladder; Lane 14, oDNA 37_{ITR}).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

The present invention provides, in a first aspect, a closed linear DNA (“clDNA”) consisting of a stem region comprising a double stranded DNA sequence of interest covalently closed at both ends by hairpin loops, the clDNA comprising at least two modified nucleotides.

As used herein, the term “closed linear DNA” or “clDNA” refers to a single stranded covalently closed DNA molecule that forms a “dumbbell” or “doggy-bone” shaped structure under conditions allowing nucleotide hybridization. Therefore, although the clDNA is formed by a single stranded DNA molecule, the formation of the “dumbbell” structure by the hybridization of two complementary sequences within the same molecule generates a structure consisting on a double-stranded middle segment flanked by two single-stranded loops. The skilled in the art knows how to generate clDNA from open or closed double stranded DNA using routine molecular biology techniques. For instance, the skilled in the art knows that a clDNA can be generated by attaching hairpin DNA adaptors—for instance, by the action of a ligase- to both ends of an open double stranded DNA. “Hairpin DNA adaptor” refers to a single stranded DNA that forms a stem-loop structure by the hybridization of two complementary sequences, wherein the stem region formed is closed at one end by a single stranded loop and is open at the other end.

The “sequence of interest” is understood as the double stranded DNA fragment that comprises the minimum necessary sequences encoding for the gene of interest together with other sequences that are required for correct gene expression, for example, an expression cassette. The sequence of interest may additionally comprise other sequences flanking the expression cassette, such as inverted terminal repeats (ITRs). The term “nucleoside” refers to a compound consisting of a base linked to the C-1′ carbon of a sugar, for example, ribose or deoxyribose. The term “nucleotide” refers to a phosphate ester of a nucleoside, as a monomer unit or within a polynucleotide.

A “modified nucleotide” is any nucleotide (e.g., adenosine, guanosine, cytidine, and thymidine) that has been chemically modified—by modification of the base, the sugar or the phosphate group- or that incorporates a non-natural moiety in its structure. Thus, the modified nucleotide may be naturally or non-naturally occurring depending on the modification.

A modified nucleotide as used herein is preferably a variant of guanosine, uridine, adenosine, thymidine and cytidine including, without implying any limitation, any naturally occurring or non-naturally occurring guanosine, uridine, adenosine, thymidine or cytidine that has been altered chemically, for example by acetylation, methylation, hydroxylation, etc., including 5-methyl-deoxycytidine, 2-amino-deoxyadenosine, 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. The preparation of such variants is known to the person skilled in the art, for example from U.S. Pat. No. 4,373,071.

The modified nucleotides may also include, without limitation pyridin-4-oneribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

The modified nucleotides may also include, without limitation 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

The modified nucleotides may also include, without limitation inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The modified nucleotides may also include, without limitation 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

The modified nucleotide may be chemically modified at the 2′ position. Preferably, the modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro.

Another chemical modification that involves the 2′ position of a nucleotide as described herein is a locked nucleic acid (LNA) nucleotide, an ethylene bridged nucleic acid (ENA) nucleotide and an (S)-constrained ethyl cEt nucleotide. These backbone modifications lock the sugar of the modified nucleotide into the preferred northern conformation. The phosphate groups of the backbone can be modified, for example, by replacing one or more of the oxygen atoms with a different substituent. Further, the modified nucleotide can include the full replacement of an unmodified phosphate moiety with a modified phosphate as described herein. Examples of modified phosphate groups include, but are not limited to, the group consisting of a phosphorothioate (also known as tiophosphate), a phosphoroselenate, a borano phosphate, a borano phosphate ester, a hydrogen phosphonate, a phosphoroamidate, an alkyl phosphonate, an aryl phosphonate and a phosphotriester. The phosphate linker can also be modified by the replacement of a linking oxygen with nitrogen (bridged phosphoroamidates), sulfur (bridged phosphorothioates) and carbon (bridged methylene-phosphonates).

The modified nucleotide may be an abasic site. As used herein, an “abasic site” is a nucleotide lacking the organic base. In preferred embodiments, the abasic nucleotide further comprises a chemical modification as described herein at the 2′ position of the ribose. Preferably, the 2′ C atom of the ribose is substituted with a substituent selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro.

In a particular embodiment of the first aspect of the invention, optionally in combination with any of the embodiments provided above or below, the at least two modified nucleotides are independently selected form the group consisting of 2-amino-deoxyadenosine, 5-methyl-deoxycytidine, thiophosphate nucleotide, LNA nucleotide, Inosine, 8-oxo-deoxyAdenosine and 5-fluoro-deoxyuracil and L-DNA nucleotide.

In a particular embodiment of the first aspect of the invention, optionally in combination with any of the embodiments provided above or below, the at least two modified nucleotides are not L-DNA nucleotide, 5-bromouridine or 5-iodouridine.

2-amino-deoxyadenosine (also known as 2-Amino-2′-deoxyadenosine or 2-Amino-dA) is a derivate from deoxyadenosine. 2-amino-deoxyadenosine has the IUPAC name (2R,3S,5R)-5-(2,6-diaminopurin-9-yl)-2-(hydroxymethyl)oxolan-3-ol, and the CAS number 4546-70-7.

5-methyl-deoxycytidine (5-Methyl-dCTP), is a derivate from deoxycytidine, which as a IUPAC name ([[(2R,3S,5R)-5-(4-amino-5-methyl-2-oxopyrimidin-1-yl)-3-hydroxyoxolan-2-yl]methoxy-hydroxyphosphoryl] phosphono hydrogen phosphate, and the CAS number 22003-12-9.

A thiophosphate nucleotide is any nucleotide that contains a thiophosphate (also known as phosphorothioate) as phosphate group. Thiophosphate has a CAS number 15181-41-6.

An LNA nucleotide is a modified RNA nucleotide in which the ribose moiety is modified with an extra bridge connecting the 2′ oxygen and 4′ carbon.

An L-DNA nucleotide refers to a nucleotide that contains the L enantiomer of the ribose or deoxyribose.

In a more particular embodiment of the first aspect of the invention, optionally in combination with any of the embodiments provided above or below, the clDNA comprises at least three, at least four, or at least five modified nucleotides independently selected form the group consisting of thiophosphate, locked nucleic acid, 2,6-diaminopurine, 5-methyl-deoxycytidine, Inosine, 8-oxo-deoxyAdenosine and 5-fluoro-deoxyuracil and L-DNA nucleotide.

In a more particular embodiment of the first aspect of the invention, optionally in combination with any of the embodiments provided above or below, the clDNA comprises two LNA nucleotides.

In a more particular embodiment of the first aspect of the invention, optionally in combination with any of the embodiments provided above or below, the at least two modified nucleotides are located in one or both single stranded end loops of the clDNA. In a more particular embodiment, at least one modified nucleotide is located in one single stranded end loop and at least another modified nucleotide is located in the other single stranded end loop.

In a more particular embodiment of the first aspect of the invention, optionally in combination with any of the embodiments provided above or below, at least one modified nucleotide is located in one of the single stranded end loops and at least another modified nucleotide is located in one of the strands forming the stem region of the clDNA.

In a more particular embodiment of the first aspect of the invention, optionally in combination with any of the embodiments provided above or below, the at least two modified nucleotides are in one or both strands forming the stem region of the clDNA.

In a more particular embodiment of the first aspect of the invention, optionally in combination with any of the embodiments provided above or below, when the at least one modified nucleotide is in one of the strands forming the stem region, the modified nucleotide is located within the strand region defined by the nucleotides at positions 1 to 5 with respect the last nucleotide forming the loop.

The nucleotide at position 1 in one of the strands forming the stem region with respect the last nucleotide forming the loop is the first nucleotide immediately after the last nucleotide of the single stranded loop; the nucleotide at position 2 in one of the strands forming the stem region with respect the last nucleotide forming the loop is the second nucleotide immediately after the last nucleotide of the single stranded loop. The same reasoning applies to the nucleotides at positions 3, 4 and 5 with respect the last nucleotide forming the loop.

In a more particular embodiment of the first aspect of the invention, optionally in combination with any of the embodiments provided above or below, when the at least one modified nucleotide is in one of the strands forming the stem region, the modified nucleotide is located within the strand region defined by the nucleotides 1 to 10 with respect to the last nucleotide forming part of the DNA sequence of interest.

The nucleotide at position 1 in one of the strands forming the stem region with respect to the last nucleotide forming part of the DNA sequence of interest is the first nucleotide immediately after the last nucleotide of the DNA sequence. The nucleotide at position 2 in one of the strands forming the stem region with respect to the last nucleotide forming part of the DNA sequence of interest is the second nucleotide immediately after the last nucleotide of the DNA sequence. The same reasoning applies to the nucleotides at positions 3-10 with respect to the last nucleotide forming part of the DNA sequence of interest.

In a particular embodiment of the first aspect of the invention, optionally in combination with any of the embodiments provided above or below, the stem region comprises two restriction sites flanking the DNA sequence of interest. In a more particular embodiment, the restriction site is selected from the group consisting of a Bsal restriction site, AfIII restriction site, HindIII restriction site, Nhel restriction site, and EcoRV restriction site. In an even more particular embodiment, the restriction site is a Bsal restriction site. The skilled in the art knows that the restriction sites can be located at any distance between the loops and the DNA sequence of interest.

In a particular embodiment of the first aspect of the invention, optionally in combination with any of the embodiments provided above or below, the clDNA comprises a primase/polymerase priming site. The primase recognition site may be present, for example, in the stem. In a particular embodiment the primase recognition site is comprised in at least one of the loops. In another particular embodiment of the first aspect of the invention, optionally in combination with any of the embodiments provided above or below, the clDNA does not comprise a primase/polymerase priming site.

By including a primase recognition site, it is facilitated the use of a primase for priming the amplification of the clDNA of the invention.

In a particular embodiment of the first aspect of the invention, optionally in combination with any of the embodiments provided above or below, the clDNA comprises inverted terminal repeats (ITR) flanking the gene of interest. In a particular embodiment the ITRs are comprised in the sequence of interest flanking an expression cassette. In another particular embodiment, the ITRs are comprised in the stem region of the adaptors. The ITRs can be at any suitable distance from the expression cassette, for instance, the ITRs can be directly linked to the expression cassette or at a distance from 1 to 50 nucleotides, from 50 to 200 nucleotides, from 200 to 1000 nucleotides. Thus, in a particular embodiment, optionally in combination with any of the embodiments provided above or below, the DNA sequence of interest comprises an expression cassette flanked by inverted terminal repeats (ITRs) at a distance from 1 to 50 nucleotides.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue.

It will be understood by one of ordinary skill in the art that in complex clDNA configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a clDNA vector is referred to as a “5′ ITR” or a “left ITR”, and an ITR located 3′ to (downstream of) an expression cassette in a clDNA vector is referred to as a “3′ ITR” or a “right ITR”.

In a particular embodiment of the first aspect of the invention, optionally in combination with any of the embodiments provided above or below, the inverted terminal repeats are of sequence SEQ ID NO: 4 or SEQ ID NO: 5.

In a particular embodiment of the first aspect of the invention, optionally in combination with any of the embodiments provided above or below, the closed linear DNA comprises a 5′ inverted terminal repeat of sequence SEQ ID NO: 4 and/or a 3′ inverted terminal repeat of sequence SEQ ID NO: 5.

In a particular embodiment the first aspect of the invention, optionally in combination with any of the embodiments provided above or below, the closed linear DNA comprises at least one DD-ITR. “DD-ITR” refers to an ITR with flanking D elements as disclosed in Xiao X. et al., “A novel 165-base-pair terminal repeat sequence is the sole cis requirement for the adeno-associated virus life cycle”, 1997, J Virol., vol. 71(2), pp. 941-948.

In a particular embodiment of the first aspect of the invention, optionally in combination with any of the embodiments provided above or below, the DNA sequence of interest comprises an expression cassette.

The term “expression cassette” refers to a DNA sequence comprising one or more promoter or enhancer elements and a gene or other coding sequence which encodes an mRNA, miRNA, siRNA or protein of interest. The expression cassette may further comprise other elements that regulate the expression of the coding sequence, such as a transcription termination site.

In a particular embodiment of the first aspect of the invention, optionally in combination with any of the embodiments provided above or below, the expression cassette comprises a eukaryotic promoter operably linked to a sequence encoding an mRNA, miRNA, siRNA or protein.

In a particular embodiment of the first aspect of the invention, optionally in combination with any of the embodiments provided above or below, the expression cassette further comprises a eukaryotic transcription termination sequence.

In a particular embodiment of the first aspect of the invention, optionally in combination with any of the embodiments provided above or below, the expression cassette lacks one or more bacterial or vector sequences selected from the group consisting of:
(i) bacterial origins of replication;
(ii) bacterial selection markers; and
(iii) unmethylated CpG motifs.

In a particular embodiment of the first aspect of the invention, optionally in combination with any of the embodiments provided above or below, the clDNA is an in vitro cell-free clDNA.

As indicated above, in a second aspect the invention provides the closed linear DNA according of the first aspect for use in therapy.

The clDNA of the invention may be used for in vitro expression in a host cell, particularly in DNA vaccines or gene therapy. DNA vaccines typically encode a modified form of an infectious organism's DNA. DNA vaccines are administered to a subject where they then express the selected protein of the infectious organism, initiating an immune response against that protein which is typically protective. DNA vaccines may also encode a tumor antigen in a cancer immunotherapy approach.

A DNA vaccine may comprise a nucleic acid sequence encoding an antigen for the treatment or prevention of a number of conditions including but not limited to cancer, allergies, toxicity and infection by a pathogen such as, but not limited to, fungi, viruses including Human Papilloma Viruses (HPV), HIV, HSV2/HSV1, Influenza virus (types A, B and C), Polio virus, RSV virus, Rhinoviruses, Rotaviruses, Hepatitis A virus, Norwalk Virus Group, Enteroviruses, Astroviruses, Measles virus, Parainfluenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Pox virus, Marburg and Ebola, SARS-CoV-1, SARS-CoV-2; bacteria including Mycobacterium tuberculosis, Chlamydia, Neisseria gonorrhoeae, Shigella, Salmonella, Vibrio cholerae, Treponema pallidum, Pseudomonas, Bordetella pertussis, Brucella, Franciscella tularensis, Helicobacter pylori, Leptospira interrogans, Legionella pneumophila, Yersinia pestis, Streptococcus (types A and B), Pneumococcus, Meningococcus, Haemophilus influenza (type b), Toxoplasma gondii, Campylobacteriosis, Moraxella catarrhalis, Donovanosis, and Actinomycosis; fungal pathogens including Candidiasis and Aspergillosis; parasitic pathogens including Taenia, Flukes, Roundworms, Amoebiasis, Giardiasis, Cryptosporidium, Schistosoma, Pneumocystis carinii, Trichomoniasis and Trichinosis.

DNA vaccines may comprise a nucleic acid sequence encoding an antigen from a member of the adenoviridae (including for instance a human adenovirus), herpesviridae (including for instance HSV-1, HSV-2, EBV, CMV and VZV), papovaviridae (including for instance HPV), poxviridae (including for instance smallpox and vaccinia), parvoviridae (including for instance parvovirus B19), reoviridae (including for instance a rotavirus), coronaviridae (including for instance SARS, including SARS-CoV-1 and SARS-CoV-2), flaviviridae (including for instance yellow fever, West Nile virus, dengue, hepatitis C and tick-borne encephalitis), picornaviridae (including polio, rhinovirus, and hepatitis A), togaviridae (including for instance rubella virus), filoviridae (including for instance Marburg and Ebola), paramyxoviridae (including for instance a parainfluenza virus, respiratory syncitial virus, mumps and measles), rhabdoviridae (including for instance rabies virus), bunyaviridae (including for instance Hantaan virus), orthomyxoviridae (including for instance influenza A, B and C viruses), retroviridae (including for instance HIV and HTLV) and hepadnaviridae (including for instance hepatitis B).

The antigen may be from a pathogen responsible for a veterinary disease and in particular may be from a viral pathogen, including, for instance, a Reovirus (such as African Horse sickness or Bluetongue virus) and Herpes viruses (including equine herpes). The antigen may be one from Foot and Mouth Disease virus, Tick borne encephalitis virus, dengue virus, SARS, West Nile virus and Hantaan virus. The antigen may be from an immunodeficiency virus, and may, for example, be from SIV or a feline immunodeficiency virus.

clDNAs produced by the process of the invention may also comprise a nucleic acid sequence encoding tumour antigens. Examples of tumour associated antigens include, but are not limited to, cancer-testes antigens such as members of the MAGE family (MAGE 1, 2, 3 etc), NY-ESO-1 and SSX-2, differentiation antigens such as tyrosinase, gp100, PSA, Her-2 and CEA, mutated self-antigens and viral tumour antigens such as E6 and/or E7 from oncogenic HPV types. Further examples of particular tumour antigens include MART-1, Melan-A, p97, beta-HCG, GaINAc, MAGE-1, MAGE-2, MAGE-4, MAGE-12, MUC1, MUC2, MUC3, MUC4, MUC18, CEA, DDC, P1A, EpCam, melanoma antigen gp75, Hker 8, high molecular weight melanoma antigen, K19, Tyr1, Tyr2, members of the pMel 17 gene family, c-Met, PSM (prostate mucin antigen), PSMA (prostate specific membrane antigen), prostate secretary protein, alpha-fetoprotein, CA125, CA19.9, TAG-72, BRCA-1 and BRCA-2 antigen.

Also, the process of the invention may produce other types of therapeutic clDNA e.g. those used in gene therapy. For example, such DNA molecules can be used to express a functional gene where a subject has a genetic disorder caused by a dysfunctional version of that gene. Examples of such diseases include Duchenne muscular dystrophy, cystic fibrosis, Gaucher's Disease, and adenosine deaminase (ADA) deficiency. Other diseases where gene therapy may be useful include inflammatory diseases, autoimmune, chronic and infectious diseases, including such disorders as AIDS, cancer, neurological diseases, cardivascular disease, hypercholestemia, various blood disorders including various anaemias, thalassemia and haemophilia, and emphysema. For the treatment of solid tumors, genes encoding toxic peptides (i.e., chemotherapeutic agents such as ricin, diptheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, may be expressed.

Other types of therapeutic clDNA are also contemplated for production by the process of the invention. For example, clDNAs which are transcribed into an active RNA form, for example a small interfering RNA (siRNA) may be produced according to the process of the invention.

In a particular embodiment of the second aspect of the invention, optionally in combination with any of the embodiments provided above or below, the clDNA is for use in DNA vaccines or gene therapy.

As mentioned above, in a third aspect the invention provides a pharmaceutical composition comprising a therapeutically effective amount of the closed linear DNA of the first aspect and pharmaceutically acceptable carriers or excipients.

The expression “therapeutically effective amount” as used herein, refers to the amount of the clDNA that, when administered, is sufficient to prevent development of, or alleviate to some extent, one or more of the symptoms of the disease which is addressed. The particular dose of agent administered according to this invention will of course be determined by the particular circumstances surrounding the case, including the clDNA administered, the route of administration, the particular condition being treated, and the similar considerations.

The expression “pharmaceutical composition” encompasses both compositions intended for human as well as for non-human animals (i.e. veterinarian compositions).

The expression “pharmaceutically acceptable carriers or excipients” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and non-human animals without excessive toxicity, irritation, allergic response, immunogenicity or other problems or complications commensurate with a reasonable benefit/risk ratio.

Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The relative amounts of the close linear DNA, the pharmaceutically acceptable excipients, and/or any additional ingredients in a pharmaceutical composition of the invention will vary, depending upon the identity, size, and/or condition of the subject treated and further depending upon the route by which the composition is to be administered.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Excipients such as coloring agents, coating agents, sweetening, and flavouring agents can be present in the composition, according to the judgment of the formulator.

The pharmaceutical compositions containing the close linear DNA produced according to the process of the invention can be presented in any dosage form, for example, solid or liquid, and can be administered by any suitable route, for example, oral, parenteral, rectal, topical, intranasal or sublingual route, for which they will include the pharmaceutically acceptable excipients necessary for the formulation of the desired dosage form, for example, topical formulations (ointment, creams, lipogel, hydrogel, etc.), eye drops, aerosol sprays, injectable solutions, osmotic pumps, etc.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, corn-starch, powdered sugar, and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and combinations thereof.

Exemplary binding excipients include, but are not limited to, starch (e.g., corn-starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminium silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, ascorbyl stearate, ascorbyl oleate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

In a fourth aspect, the present invention provides a process for the production of a clDNA comprising at least two modified nucleotides according to the first aspect, comprising the steps of a) providing a DNA template comprising a DNA sequence of interest; b) amplifying DNA from the DNA template of step (a) producing a concatameric DNA comprising repeats of the DNA sequence of interest, wherein each one of the repeated DNA sequences of interest is flanked by restriction sites; c) generating a closed linear DNA with the amplified DNA produced in step (b) by (c.1) contacting the concatameric DNA with at least one restriction enzyme thereby producing a plurality of open double stranded DNA fragments each containing the DNA sequence of interest, and (c.2) attaching a hairpin DNA adaptor at each one of the ends of the open double stranded DNA fragments, wherein each one of the adaptors has at least one modified nucleotide or, alternatively, only one of the adaptors attached to the DNA fragment comprises the at least two modified nucleotide, and d) purifying the closed linear DNA produced in step (c).

In a particular embodiment of the fourth aspect of the invention, optionally in combination with any of the embodiments provided above or below, the hairpin DNA adaptor is from 6 to 600 nucleotides in length. In a more particular embodiment, the hairpin DNA adaptor is from 6 to 200 nucleotides in length. In a more particular embodiment, the adaptor is from 6 to 60 nucleotides in length. In another particular embodiment, the adaptor is from 10 to 60 nucleotides in length. In another particular embodiment, the adaptor is from 10 to 40 nucleotides in length.

In a particular embodiment of the method of the fourth aspect, optionally in combination with any of the methods provided above or below, the amplification is primed with random primers or with a primase/polymerase enzyme.

The amplification of the DNA template using a primase/polymerase as a priming enzyme, generates amplified DNA with very high efficiency and fidelity, which can be later processed to generate closed linear DNA suitable for therapeutic uses.

As used herein, the term “priming” refers to the generation of an oligonucleotide primer on a polynucleotide template.

The term “primase/polymerase enzyme” refers to a DNA-directed primase/polymerase enzyme, such as the enzymes from the archaeo-eukaryotic primase (AEP) superfamily. These enzymes present the capacity of starting DNA chains with dNTPs. Enzymes from this superfamily that can be used in the invention are, for example, Thermus thermophilus primase/polymerase (TthPrimPol) or human primase/polymerase (hsPrimPol, CCDC111, FLJ33167, EukPrim2 or hPrimPol1). “Thermus thermophilus primase/polymerase” or “TthPrimPol” refers to the primase/polymerase of the bacteria Thermus thermophilus of sequence SEQ ID NO: 1. The nucleotide and protein sequences are available in the NCBI Entrez database as NC_005835 and WP_01 1173100.1, respectively.

TABLE 1

| SEQ ID | Name | Sequence |
|---|---|---|
| SEQ ID | TthPrimPol | MRPIEHALSYAAQGYGVLPLRPGGKEPLGKLVPHGLKNASR |
| NO: 1 | | DPATLEAWWRSCPRCGVGILPGPEVLVLDFDDPEAWEGLR |
| | | QEHPALEAAPRQRTPKGGRHVFLRLPEGVRLSASVRAIPGV |
| | | DLRGMGRAYVVAAPTRLKDGRTYTWEAPLTPPEELPPVPQA |
| | | LLLKLLPPPPPPRPSWGAVGTASPKRLQALLQAYAAQVARTP |
| | | EGQRHLTLIRYAVAAGGLIPHGLDPREAEEVLVAAAMSAGLP |
| | | EWEARDAVRWGLGVGASRPLVLESSSKPPEPRTYRARVYA |
| | | RMRRWV |
| SEQ ID | TelN | MSKVKIGELINTLVNEVEAIDASDRPQGDKTKRIKAAAARYKN |
| NO: 2 | | ALFNDKRKFRGKGLQKRITANTFNAYMSRARKRFDDKLHHS |
| | | FDKNINKLSEKYPLYSEELSSWLSMPTANIRQHMSSLQSKLK |
| | | EIMPLAEELSNVRIGSKGSDAKIARLIKKYPDWSFALSDLNSD |
| | | DWKERRDYLYKLFQQGSALLEELHQLKVNHEVLYHLQLSPA |
| | | ERTSIQQRWADVLREKKRNVVVIDYPTYMQSIYDILNNPATLF |
| | | SLNTRSGMAPLAFALAAVSGRRMIEIMFQGEFAVSGKYTVNF |
| | | SGQAKKRSEDKSVTRTIYTLCEAKLFVELLTELRSCSAASDF |
| | | DEVVKGYGKDDTRSENGRINAILAKAFNPWVKSFFGDDRRV |
| | | YKDSRAIYARIAYEMFFRVDPRWKNVDEDVFFMEILGHDDEN |
| | | TQLHYKQFKLANFSRTWRPEVGDENTRLVALQKLDDEMPGF |
| | | ARGDAGVRLHETVKQLVEQDPSAKITNSTLRAFKFSPTMISR |
| | | YLEFAADALGQFVGENGQWQLKIETPAIVLPDEESVETIDEP |
| | | DDESQDDELDEDEIELDEGGGDEPTEEEGPEEHQPTALKPV |
| | | FKPAKNNGDGTYKIEFEYDGKHYAWSGPADSPMAAMRSAW |
| | | ETYYS |
| SEQ ID | TelN target | TATCAGCACACAATTGCCCATTATACGCGCGTATAATGGA |
| NO: 3 | sequence | CTATTGTGTGCTGATA |
| SEQ ID | 5′ ITR | CCTGCAGGCAGCTGCGCGCTCGCTCGCTCACTGAGGCCG |
| NO: 4 | sequence | CCCGGGCAAAGCCCGGGCGTCGGGCGACCTTTGGTCGC |
| | | CCGGCCTCAGTGAGCGAGCGAGCGCGCAGAGAGGGAGT |
| | | GGCCAACTCCATCACTAGGGGTTCCT |
| SEQ ID | 3′ ITR | AGGAACCCCTAGTGATGGAGTTGGCCACTCCCTCTCTGC |
| NO: 5 | sequence | GCGCTCGCTCGCTCACTGAGGCCGGGCGACCAAAGGTC |
| | | GCCCGACGCCCGGGCTTTGCCCGGGCGGCCTCAGTGAG |
| | | CGAGCGAGCGCGCAGCTGCCTGCAGG |

In a particular embodiment of the process of the fourth of the invention, optionally in combination with any of the embodiments provided above or below, the amplification of step (b) is primed with a primase/polymerase enzyme selected from TthPrimPol or hsPrimPol. In a particular embodiment, the primase polymerase enzyme is TthPrimPol. In a more particular embodiment, the primase polymerase enzyme is TthPrimPol of SEQ ID NO: 1 or a variant thereof which has a sequence identity of at least 80%, at least 85%, at least 90%, or at least 95% with respect to SEQ ID NO: 1. The skilled in the art would know that any variant of TthPrimPol which maintains its primase activity would be suitable for use in the process of the invention.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, among others. Preferred software analysis programs include the ALIGN, CLUSTAL W, and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes, or PAM matrixes (e.g., the PAM30, PAM70, PAM120, PAM160, PAM250, and PAM350 matrixes), are used in determining identity.

The BLAST programs provide analysis of at least two amino acid sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations. If gap existence costs (or gap scores) are used, the gap existence cost preferably is set between about −5 and −15. Similar gap parameters can be used with other programs as appropriate. The BLAST programs and principles underlying them are further described in, e.g., Altschul et al., “Basic local alignment search tool”, 1990, J. Mol. Biol, v. 215, pages 403-410. A particular percentage of identity encompasses variations of the sequence due to conservative mutations of one or more amino acids leading to a TthPrimPol enzyme being still effective, thus able to prime suitable sequences. Protein variations are also due to insertions or deletions of one or more amino acids.

In a particular embodiment of the process of the fourth aspect of the invention, optionally in combination with any of the embodiments provided above or below, the process is an in vitro cell-free process for the production of closed linear DNA.

In a particular embodiment of the process of the fourth aspect of the invention, optionally in combination with any of the embodiments provided above or below, the amplification of step (b) is a rolling-circle amplification.

The term “rolling-circle amplification” or “RCA” refers to nucleic acid amplification reactions involving the amplification of covalently closed DNA molecules, such as clDNA or double stranded circular DNA, wherein a polymerase performs the extension of a primer around the closed DNA molecule. The polymerase displaces the hybridized copy and continues polynucleotide extension around the template to produce concatameric DNA comprising tandem units of the amplified DNA. These linear single stranded products serve as the basis for multiple hybridization, primer extension and strand displacement events, resulting in formation of concatameric double stranded DNA products. There are thus multiple copies of each amplified single unit DNA in the concatameric double stranded DNA products The skilled in the art knows, making use of their general knowledge and/or the instructions of the manufacturer, how to adjust the conditions of the amplification step depending on the enzymes and the characteristics of the template to be amplified. Depending on how the template DNA is generated, the concatameric DNA will contain different sequences flanking each amplified DNA sequence of interest. For example, in the concatameric DNA the repeated DNA sequence of interest may be flanked by restriction sites, protelomerase target sequences, recombinase recognition sites, or any combination thereof.

In a particular embodiment of the process of the first aspect of the invention, optionally in combination with any of the embodiments provided above or below, the amplification of step (b) is carried out with a strand displacement DNA polymerase. The term “strand-displacement DNA polymerase” refers to a DNA polymerase that that performs a 3′ end elongation reaction while removing a double-stranded portion of template DNA. Strand displacement DNA polymerases that can be used in the present invention may not be particularly limited, as long as they have such a strand-displacement activity, such as phi29 DNA polymerase and Bst DNA polymerase. Depending on the thus selected polymerase type, the skilled in the art would know that the reaction conditions for a 3′ end elongation reaction may be adequately set. For example, when phi29 DNA polymerase is used, a reaction may be performed at an optimum temperature for the reaction from 25° C. to 35° C.

Thus, in a particular embodiment, the strand displacement DNA polymerase is selected from the group consisting of phi29 DNA polymerase, Bst DNA polymerase, Bca (exo-) DNA polymerase, Klenow fragment of Escherichia coli DNA polymerase I, Vent (Exo-) DNA polymerase, DeepVent (Exo-) DNA polymerase, and KOD DNA polymerase. In a more particular embodiment, the strand displacement DNA polymerase is phi29 DNA polymerase. In an even more particular embodiment, the strand displacement DNA polymerase is a chimeric protein comprising a phi29 DNA polymerase. The skilled in the art knows how to obtain chimeric DNA polymerases with improved characteristics, for example as disclosed in WO2011000997.

In a particular embodiment of the process of the fourth aspect of the invention, optionally in combination with any of the embodiments provided above or below, the DNA template is selected from a closed linear DNA template or a circular double stranded DNA template.

As use herein, the term “circular double stranded DNA” refers to a covalently closed double stranded DNA molecule.

In a particular embodiment of the process of the fourth aspect of the invention, optionally in combination with any of the embodiments provided above or below, step (a) is performed by:
contacting a plasmid vector comprising at least two restriction sites flanking the DNA sequence of interest with at least one restriction enzyme thereby producing open double stranded DNA containing the DNA sequence of interest, and attaching hairpin DNA adaptors to both ends of the open double stranded DNA containing the DNA sequence of interest; or, alternatively, it is performed by:
contacting a plasmid vector comprising at least two protelomerase target sequences flanking the DNA sequence of interest with a protelomerase, more particularly, with TeIN; thus, obtaining a DNA template which is a closed linear DNA template containing the DNA sequence of interest.

As used herein, a “plasmid vector” refers to a circular double stranded nucleic acid molecule capable of transporting another nucleic acid to which it has been linked and which is capable of autonomous replication withing a cell independently of the chromosomal DNA. Therefore, plasmid vectors contain all the elements needed for replication in a cell, particularly, in a bacterial cell.

The use of restriction enzymes and ligases (for attaching) is routinely in the field of molecular biology, therefore the skilled in the art would know how to adjust the conditions of the reaction depending on the enzymes used, and which restriction enzyme should be used depending on the restriction site to be targeted.

The skilled in the art also knows that some restriction enzymes generate DNA overhangs (sticky ends) while others do not (blunt ends). Both types of restriction enzymes can be used in the method of the invention. The skilled man knows that an adaptor with sticky ends can be attached to an open double stranded DNA with sticky ends (sticky-end ligation). An open double stranded DNA with blunt ends can also be dA-tailed by a process of adding a terminal 3′deoxy adenosine nucleotide, for instance using Taq polymerase, and then ligated to an adaptor with an overhanging T.

In a particular embodiment of the process of the fourth aspect of the invention, optionally in combination with any of the embodiments provided above or below, the restriction enzyme generates blunt ends or sticky ends. In a more particular embodiment, the contacting a plasmid vector comprising at least two restriction sites flanking the DNA sequence of interest with at least one restriction enzyme produces open double stranded DNA with sticky ends or open double stranded DNA with blunt ends.

The adaptors attached to both ends of the open double stranded DNA to form de clDNA can be the same adaptor or different adaptors.

In a particular embodiment of the process of the fourth aspect of the invention, optionally in combination with any of the embodiments provided above or below, the hairpin DNA adaptors comprise at least one restriction site. In a more particular embodiment, the restriction site is selected from the group consisting of a Bsal restriction site, AfIII restriction site, HindIII restriction site, Nhel restriction site, and EcoRV restriction site. In an even more particular embodiment, the restriction site is a Bsal restriction site. In another particular embodiment the restriction site is selected from Bbsl and BseRI restriction sites.

In a particular embodiment of the process of the fourth aspect of the invention, optionally in combination with any of the embodiments provided above or below, the hairpin DNA adaptors do not contain a primase recognition site. In a more particular embodiment, the hairpin DNA adaptors do not contain the sequence XTC.

In a more particular embodiment, optionally in combination with any of the embodiments provided above or below, the hairpin DNA adaptors contain a protelomerase target sequence. In an even more particular embodiment, the hairpin DNA adaptors contain a portion of a protelomerase target sequence.

As used herein, “protelomerase” is any polypeptide capable of cleaving and rejoining a template comprising a protelomerase target site in order to produce a covalently closed linear DNA molecule. Thus, the protelomerase has DNA cleavage and ligation functions. Enzymes having protelomerase-type activity have also been described as telomere resolvases (for example in Borrelia burgdorferi). A typical substrate for protelomerase is circular double stranded DNA. If this DNA contains a protelomerase target site, the enzyme can cut the DNA at this site and ligate the ends to create a linear double stranded covalently closed DNA molecule. The ability of a given polypeptide to catalyze the production of closed linear DNA from a template comprising a protelomerase target site can be determined using any suitable assay described in the art.

Examples of suitable protelomerases for use in the process of the invention include those from bacteriophages such as phiHAP-1 from Halomonas aquamarina, PY54 from Yersinia enterolytica, phiKO2 from Klebsiella oxytoca and VP882 from Vibrio sp., and N15 from Escherichia coli, or variants of any thereof.

In a particular embodiment of the process of the first aspect of the invention, optionally in combination with any of the embodiments provided above or below, the protelomerase is bacteriophage N15 TeIN of SEQ ID NO: 2 or a variant thereof which comprises a sequence having at least 80% identity to SEQ ID NO: 2.

A “protelomerase target sequence” is any DNA sequence whose presence in a DNA template allows for its conversion into a closed linear DNA by the enzymatic activity of protelomerase. In other words, the protelomerase target sequence is required for the cleavage and religation of double stranded DNA by protelomerase to form covalently closed linear DNA. Typically, a protelomerase target sequence comprises any perfect palindromic sequence i.e. any double-stranded DNA sequence having two-fold rotational symmetry, also described herein as a perfect inverted repeat.

In a particular embodiment of the process of the first aspect of the invention, optionally in combination with any of the embodiments provided above or below, at least two protelomerase target sequences comprises a perfect inverted repeat DNA sequence.

In a particular embodiment of the process of the first aspect of the invention, optionally in combination with any of the embodiments provided above or below, the protelomerase target sequence comprises the sequence of SEQ ID NO: 3 or a variant thereof which comprises a sequence having at sequence identity of at least 80%, at least 85%, at least 90%, or at least 95% sequence identity with respect to SEQ ID NO: 3.

The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g. E. coli N15, the central perfect inverted palindrome is flanked by inverted repeat sequences, i.e. forming part of a larger imperfect inverted palindrome.

A protelomerase target sequence as used in the invention preferably comprises a double stranded palindromic (perfect inverted repeat) sequence of at least 14 base pairs in length.

The perfect inverted repeat may be flanked by additional inverted repeat sequences. The flanking inverted repeats may be perfect or imperfect repeats i.e. may be completely symmetrical or partially symmetrical. The flanking inverted repeats may be contiguous with or non-contiguous with the central palindrome. The protelomerase target sequence may comprise an imperfect inverted repeat sequence which comprises a perfect inverted repeat sequence of at least 14 base pairs in length

A protelomerase target sequence comprising the sequence of SEQ ID NO: 3 or a variant thereof is preferred for use in combination with E. coli N15 TeIN protelomerase of SEQ ID NO: 2 and variants thereof.

Variants of any of the palindrome or protelomerase target sequences described above include homologues or mutants thereof. Mutants include truncations, substitutions or deletions with respect to the native sequence. A variant sequence is any sequence whose presence in the DNA template allows for its conversion into a closed linear DNA by the enzymatic activity of protelomerase. This can readily be determined by use of an appropriate assay for the formation of closed linear DNA. Any suitable assay described in the art may be used. Preferably, the variant allows for protelomerase binding and activity that is comparable to that observed with the native sequence. Examples of preferred variants of palindrome sequences described herein include truncated palindrome sequences that preserve the perfect repeat structure, and remain capable of allowing for formation of closed linear DNA. However, variant protelomerase target sequences may be modified such that they no longer preserve a perfect palindrome, provided that they are able to act as substrates for protelomerase activity.

It should be understood that the skilled person would readily be able to identify suitable protelomerase target sequences for use in the invention on the basis of the structural principles outlined above. Candidate protelomerase target sequences can be screened for their ability to promote formation of closed linear DNA using the assays described above.

In a particular embodiment of the process of the fourth aspect of the invention, optionally in combination with any of the embodiments provided above or below, when the DNA template is a circular double stranded DNA template containing the DNA sequence of interest, then step (a) is performed by contacting a plasmid vector comprising at least two recombinase recognition sites flanking the DNA sequence of interest with a site-specific recombinase, more particularly, a Cre recombinase.

The action of the site-specific recombinase on the plasmid vector triggers the recombination of the two recombinase recognition sites thereby generating a smaller circular double stranded DNA that contains the DNA sequence of interest that was located between the recombinase recognition sites in the plasmid vector.

“Site-specific recombinase” as used herein refers to a family of enzymes that mediate the site-specific recombination between specific DNA sequences recognized by the enzymes known as recombinase recognition sites. Examples of site-specific recombinases include, without limitation, Cre recombinase, Flp recombinase, the lambda integrase, gamma-delta resolvase, Tn3 resolvase, Sin resolvase, Gin invertase, Hin invertase, Tn5044 resolvase, Tn3 transposase, sleeping beauty transposase, IS607 transposase, Bxb I integrase, wBeta integrase, BL3 integrase, phiR4 integrase, AII 8 integrase, TGI integrase, MRU integrase, phi370 integrase, SPBc integrase, SV1 integrase, TP901-1 integrase, phiRV integrase, FC1 integrase, K38 integrase, phiBTI integrase and phiC31 integrase.

“Recombinase recognition sites” refers to nucleotide sequences that are recognized by a site-specific recombinase and can serve as a substrate for a recombination event. Non-limiting examples of recombinase recognition sites include FRT, FRT11, FRT71, attp, att, rox, and lox sites such as loxP, lox511, 1oχ2272, 1oχ66, 1oχ71, loxM2, and lox5171.

The skilled in the art would know, using his common general knowledge, that each site-specific recombinase recognizes a particular recombinase recognition site, thus depending on the recognition sequence contained in the plasmid vector a different recombinase should be used for generating the circular double stranded DNA template from the plasmid vector.

In a particular embodiment of the process of the fourth aspect of the invention, optionally in combination with any of the embodiments provided above or below, the site-specific recombinase is Cre recombinase. In a more particular embodiment, the recombinase recognition site is loxP. In an even more particular embodiment, the site-specific recombinase is Cre recombinase and the recombinase recognition site is loxP.

Thus, in a particular embodiment of the process of the fourth aspect of the invention, optionally in combination with any of the embodiments provided above or below, the amplified DNA resulting from step (b) is a concatameric DNA comprising repeats of the DNA sequence of interest, wherein each one of the repeated DNA sequences of interest is flanked by restriction sites, protelomerase target sequences, and/or recombinase recognition sites.

The skilled in the art knows that if a restriction enzyme is used to produce the template clDNA, the same restriction enzyme can be later used to generate clDNA from the amplified DNA produced in step (b). The hairpin DNA adaptors used in step (a) for generating the template clDNA can be same or different to the ones used in step (c). The adaptors are preferably different.

In a particular embodiment of the process of the first aspect of the invention, optionally in combination with any of the embodiments provided above or below, the process is for the production of a closed linear expression cassette DNA.

In a particular embodiment of the process of the fourth aspect of the invention, optionally in combination with any of the embodiments provided above or below, step (a) is performed by contacting a plasmid vector comprising at least two restriction sites flanking the DNA sequence of interest with at least one restriction enzyme thereby producing open double stranded DNA containing the DNA sequence of interest, and attaching hairpin DNA adaptors to both ends of the open double stranded DNA containing the DNA sequence of interest; and step (c) is performed by (c.1) contacting the concatameric DNA with at least one restriction enzyme thereby producing a plurality of open double stranded DNA fragments each containing the DNA sequence of interest, and (c.2) attaching the hairpin DNA adaptors as defined in the first aspect of the invention to both ends of the open double stranded DNA fragments. In a more particular embodiment, the restriction enzyme generates sticky ends or blunt ends. When the restriction enzyme generates blunt ends, the resulting fragment can be attached to adaptors containing blunt ends or alternatively it can be dA-tailed, as explained above, and then attached to an adaptor with an overhanging T.

In a particular embodiment of the process of the fourth aspect of the invention, optionally in combination with any of the embodiments provided above or below, when the DNA template is a circular double stranded DNA template containing the DNA sequence of interest flanked by restriction sites, then step (a) is performed by contacting a plasmid vector comprising at least two recombinase recognition sites flanking at least two restriction sites flanking the DNA sequence of interest with a site-specific recombinase, more particularly, a Cre recombinase; and step (c) is performed by (c.1) contacting the concatameric DNA with at least one restriction enzyme thereby producing a plurality of open double stranded DNA fragments each containing the DNA sequence of interest, and (c.2) attaching hairpin DNA adaptors as defined in the first aspect to both ends of the open double stranded DNA fragments.

In a particular embodiment of the process of the first aspect of the invention, optionally in combination with any of the embodiments provided above or below, step (a) is performed by contacting a plasmid vector comprising at least two protelomerase target sequences flanking at least two restriction sites flanking the DNA sequence of interest with a protelomerase, more particularly, with TeIN; and step (c) is performed by (c.1) contacting the concatameric DNA with at least one restriction enzyme thereby producing a plurality of open double stranded DNA fragments each containing the DNA sequence of interest, and (c.2) attaching hairpin DNA adaptors according to the first aspect to both ends of the open double stranded DNA fragments.

In a fifth aspect, the invention provides a closed linear DNA obtainable by the process according to the fourth aspect.

The seventh and eight aspects are referred to compositions comprising the clDNA of the invention containing at least two modified oligonucleotides and a carrier. The carrier may be a viral or non-viral vector. A “viral vector” is a modified virus that serves as a vehicle for introducing exogenous genetic material into the nucleus of a cell. In the sense of the present invention “non-viral vector” is any substance other that virus-derived which serves as carrier to deliver the clDNA. Non-viral vectors include nanoparticles, liposomes, vesicles and polymers.

When the non-viral vector is a polymer, for example, a polycationic polymer, the complex formed by the clDNA and the polymer is called “polyplex”. “Polyplexes” are formed by electrostatic interaction between DNA and cationic polymers (catiomers) and have attracted much attention as a safe, versatile alternative to viral vectors.

Particularly suitable polymers in the sense of the present invention are polycationic polymers, such as those disclosed in EP1859812. Some of these polymers are polyethylene glycol-based polycationic polymers. In a particular embodiment, the polyplex of the eight aspect of the invention contains the polymer with formula I.

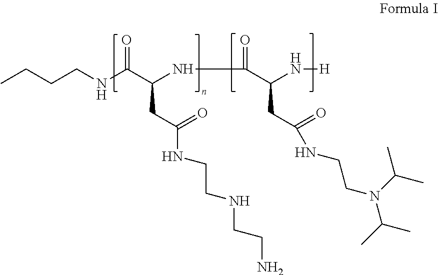

The invention also provides the compositions comprising the clDNA of the invention and a carrier, or the polyplexes, as defined above, for use in therapy or diagnosis.

For the purposes of the invention the expressions “obtainable”, “obtained” and equivalent expressions are used interchangeably, and in any case, the expression “obtainable” encompasses the expression “obtained”. All the embodiments provided under the first and fourth aspects of the invention are also embodiments of the closed linear DNA of the fifth aspect of the invention.

In a sixth aspect, the invention provides a kit for the production of clDNA comprising hairpin DNA adaptors containing at least one modified nucleotide, a ligase, and optionally, instructions for its use.

This kit can be used to manufacture the clDNA of the invention by ligation the adaptors therein provided to any given DNA sequence of interest through the action of the ligase enzyme. All the embodiment concerning the adaptors of the fourth aspect of the invention are also meant to apply to the adaptors of the kit of the sixth aspect of the invention.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

### Example 1. Synthesis of clDNAs with at Least Two Modified Nucleotides

### Synthesis of Hairpin DNA Adaptors

Hairpin DNA adaptors were synthesized following standard phosphoramidite chemistry (Beaucage S. L. et al, 1981) including at least two of the following modified nucleotides: 8-oxo-deoxyadenosine (8-oxo-dA), 5-Fluoro-deoxyuracil (5FU), inosine, thiophosphate nucleotide, or locked nucleic acid (LNA) nucleotide.

Briefly, Phophoramidite synthesis begins with the 3′-most nucleotide and proceeds through a series of cycles composed of fours steps that are repeated until the 5′-most nucleotide is attached. These steps are deprotection(i), coupling(ii), oxidation(iii), and capping(iv).

This cycle is repeated for each nucleotide in the sequence. At the end of the synthesis the oligonucleotide exists as, for example, a 25-mer with the 3′ end still attached to the CPG and the 5′ end protected with a trityl group. In addition, protecting groups remain on three of the four bases to maintain the integrity of the ring structures of the bases. The protecting groups are benzoyl on A and C and N-2-isobutyryl on G. Thymidine needs no protecting group. The completed synthesis is detritylated and then cleaved off the controlled pore glass leaving a hydroxyl on both the 3′ and 5′ ends. At this point the oligo (base and phosphate) is deprotected by base hydrolysis using ammonium hydroxide at high temperature. The final product is a functional single-stranded DNA molecule.

TABLE 2
Protected nucleotides and modified nucleotides for synthesis of hairpin DNA
adaptors. 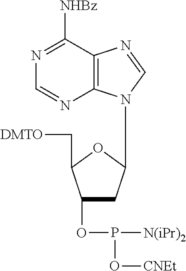
AC- dC-CE Phosphoramidite 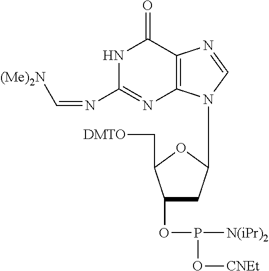
dmf-dG-CE Phosphoramidite 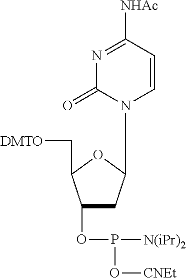
dA-CE Phosphoramidite 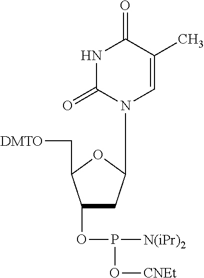
dT-CE Phosphoramidite 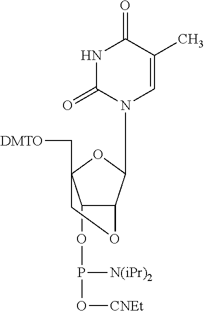
T-LNA 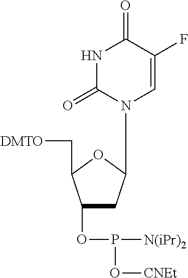
5-F-dU-CE Phosphoramidite 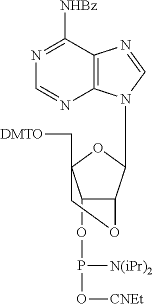
Bz-A-LNA 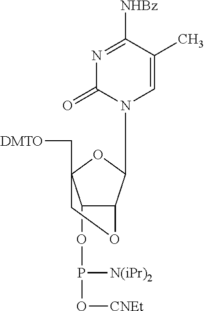
5-Me-Bz-C-LNA 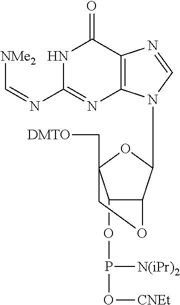
dmf-G-LNA 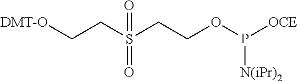
Phosphate Amidite 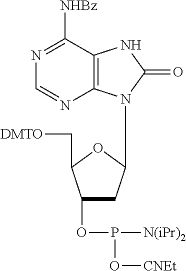
8-oxo-dA-CE Phosphoramidite 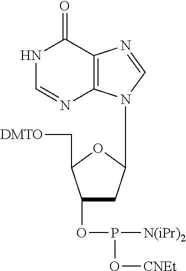
dl-CE Phosphoramidite 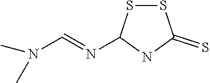
DDTT

Corresponding hairpin DNA adaptors containing natural oligonucleotides were also synthesized for comparison. The list of synthesized adaptors is provided in table 3.

Upon synthesis completion, the oligonucleotides were cleaved from the support and the protecting groups removed, standard purification step (e.g. PAGE, HPLC and/or RNase Free HPLC) was then employed to separate the full-length product from the truncated sequences.

TABLE 3
Hairpin DNA adaptors containing natural and modified nucleotides

| Sample Name | Oligo sequence | SEQ ID |
|---|---|---|
| Oligo 15 | AGGGATCCACTCAGGAT | SEQ ID NO: 6 |
| Oligo 37 | AGGGATCC*A*C*T*C*AGGAT | SEQ ID NO: 7 |
| Oligo 4 | AGGGCTAACCACTCAGGTTAG | SEQ ID NO: 8 |
| Oligo 28 | AGGGCTAACCXCTCXGGTTAG | SEQ ID NO: 9 |
| Oligo 29 | AGGGCTAACCA/i5F-dU/T/i5FdU/AGGTTAG | SEQ ID NO: 10 |
| Oligo 17 | AGGGATAACATGGCCACTCAGGCCATGTTAT | SEQ ID NO: 11 |
| Oligo 19 | AGGGATAACA+T+G+G+C+CACTCAGGCCATGTTAT | SEQ ID NO: 12 |
| Oligo 22 | AGGGATAACATGGCC/i8-oxo-dA/CTC/i8-oxo-dA/GGCCATGTTAT | SEQ ID NO: 13 |
| Oligo 21 | AGGGATAACATGGCC/I/CTC/I/GGCCATGTTAT | SEQ ID NO: 14 |
| Oligo 41 | AGGGCTTACG*C*G*C*GTAAG | SEQ ID NO: 15 |

*, phosphothioated nucleotide to the right (eg Oligo 37, phosphothioated nucleotides are: ACTCA)+, LNA nucleotide to the right (eg Oligo 19, LNA nucleotides are: TGGCC)
/I/ inosine nucleotide
i8-oxo-dA, 8-oxo-deoxyadenosine nucleotide
i5F-dU, 5-Fluoro-deoxyuracil nucleotide

### Preparation of clDNA Containing Modified Oligonucleotides

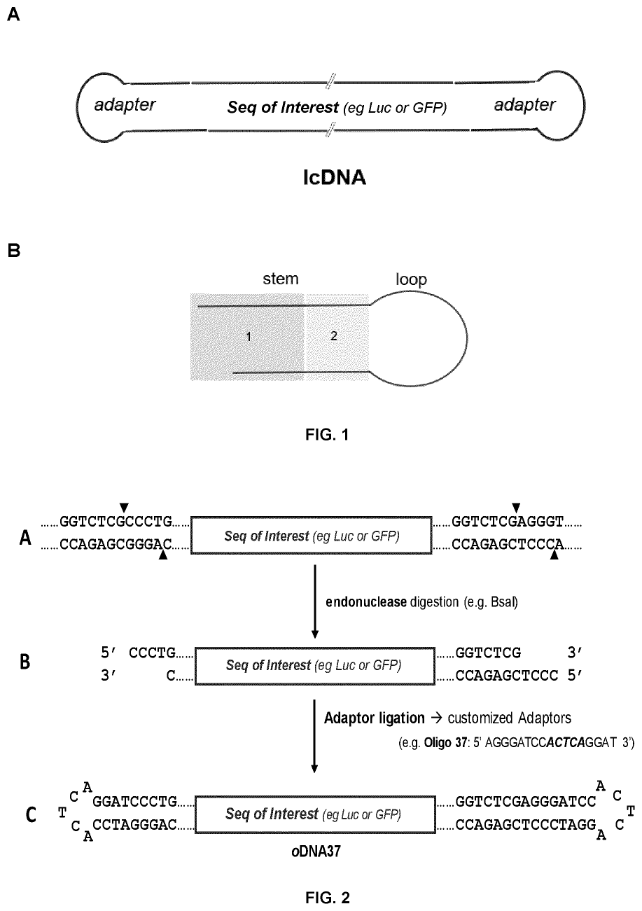

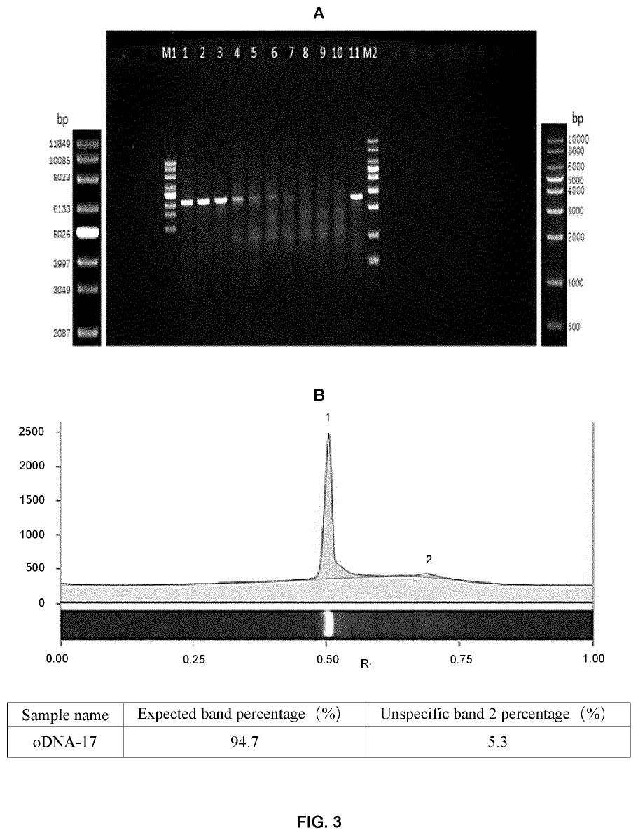

Then, clDNAs were prepared by attaching the hairpin DNA adaptors of SEQ ID NOs: 6 to 15 obtained in the section above to a double stranded DNA fragment comprising the sequence of interest by the action of a ligase (see FIG. 1). FIG. 2 shows the preparation scheme for clDNAs prepared with the hairpin adaptors described above. As shown in the figure, a DNA fragment comprising the sequence of interest flanked at each side by endonuclease restriction sites (A), was treated with the specific restriction endonuclease (B) and ligated with the desired hairpin adaptors (C).

The Sequence of Interest in these particular examples comprised the sequence encoding for luciferase enzyme (for ligating with adaptors of SEQ ID Nos: 6-13) or green fluorescent protein (GFP) (for ligating with adaptors of SEQ ID Nos: 14 and 15) flanked by restriction sites, in these examples, Bsal restriction sites. Thus, for all the constructions prepared in the present example, the DNA fragment to which the hairpin adaptors were attached comprised the sequence encoding for luciferase or GFP (together with additional sequences such as corresponding promoter and enhancer) flanked on both sides by Bsal overhangs. In FIG. 2, the exemplified hairpin adaptors on each side are Oligo 37 (SEQ ID NO: 7), which contains 5 phosphothioated nucleotides (shown in italics in FIG. 2). After adaptor ligation the samples were treated with exonuclease, endotoxin was removed with Triton-114 and purified.

This scheme applies for all clDNAs in the examples, each obtained by attaching the different hairpin adaptors (Oligos with SEQ ID NO: 6 to 15) to a double stranded DNA fragment comprising the sequence encoding for luciferase or Gfp (together with additional sequences such as corresponding promoter and enhancer) flanked on both sides by Bsal overhangs as shown in FIG. 2. Each clDNA contained the same hairpin adaptor on both sides of the double stranded DNA fragment. The resulting clDNAs are named oDNA and numbered after the hairpin adaptors used for their preparation (see table 3), that is: oDNA 15, oDNA 37, oDNA 4, oDNA 28, oDNA29, oDNA17, oDNA19, oDNA22, oDNA 21 and oDNA 41. oDNA 37, oDNA 28, oDNA29, oDNA19, oDNA22, oDNA 21 and oDNA 41 contain modified nucleotides. oDNA 15 is the natural counterpart of oDNA 37. oDNA 4 is the natural counterpart of oDNA 28 and oDNA 29. oDNA 17 is the natural counterpart of oDNA 19 and oDNA 22.

Protocol for Preparing clDNA with Customized Adaptors

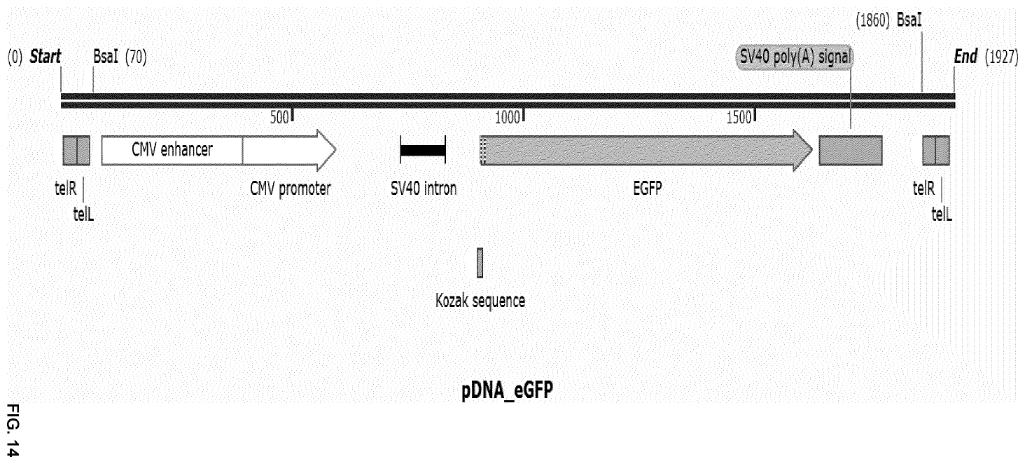

An example of a particular protocol that may be followed to obtain the above oDNAs is provided below. This protocol illustrates the preparation of clDNAs that may contain modified nucleotides (oDNAs) starting from a plasmid DNA (pDNA). Briefly, the pDNA, for example, the eGFP plasmid of SEQ ID NO: 16 comprising the sequence of interest (which in turn comprises the sequence encoding for GFP together with additional sequences such as corresponding promoter and enhancer) flanked by Bsal restriction sites and as well as protelomerase target sequences (see FIG. 14), was treated with protelomerase to yield clDNA comprising the sequence of interest flanked by endonuclease restriction sites. This clDNA was amplified via rolling circle amplification (RCA) using TthPrimPol and Phi29. The resulting concatamers were purified and treated with the corresponding restriction enzyme (eg BSal) and ligated with the hairpin adaptors containing modified nucleotides.

A. Protocol for Obtaining clDNA from Plasmid DNA

TABLE 4
Summary of experimental instruments

| Instrument | Brand/manufacturer | Model |
|---|---|---|
| Balance | Mettler Toledo | ME4002E |
| pH meter | INSEA | PHSJ-5 |
| Centrifuge | ThermoFisher | Heraeus ™ |
| | | Pico ™ 21 |
| Clean bench | AIRTECH | SW-CJ-2FD |

TABLE 5
Summary of material information

| No. | Material name | Brand or Manufacturer | Cat. No. |
|---|---|---|---|
| 1 | Exonuclease III | NEB | M0206 |
| 2 | NEBuffer 1 | NEB | M0206 |
| 3 | TritonX-114 | Solarbio | T8210 |
| 4 | Isopropyl alcohol | Sinopharm Chemical | 67-63-0 |
| | | Reagent Co., Ltd. | |
| 5 | KpnI | NEB | R3142L |
| 6 | HindIII | NEB | R3104S |
| 7 | CutSmart buffer | NEB | B7204S |
| 8 | TelN | GenScript | NA |
| 9 | TelN buffer | GenScript | NA |

### 1.1 TeIN Digestion

The eGFP plasmid was digested by TeIN enzyme at 30° C. for 2 h and inactivated at 75° C. for 10 min. Scaling up accordingly when performing several reactions at the same time.

TABLE 6
TelN enzyme digestion reaction

| COMPONENTS | 20 mL of REACTION |
|---|---|
| 10X Buffer | 2 mL |
| Plasmid | 10 mg, 10 mL |
| TelN | Actual addition: 1.0 × 10⁶ U(2 mL, 50 U/μL) |
| Sterile water | Add to 20 mL |

### 1.2 Backbone Removal

1.2.1 Kpn I and Hind III Digestion

The product from last step was digested with Kpn I and Hind Ill at 37° C. for 1 h. Then the sample was inactivated at 65° C. for 15 minutes. Scaling up accordingly when performing several reactions at the same time.

TABLE 7
Kpn I and Hind III digestion reaction

| COMPONENTS | 25 mL of REACTION | |
|---|---|---|
| 10x Cutsmart | 2.5 | mL |
| Plasmid from last step | 20 | ml |

| | |
|---|---|
| Kpn I | Actual addition: 10000 U |
| | (500 μL, 20 U/μL) |
| Hind III | Actual addition: 10000 U |
| | (500 μL, 20 U/μL) |
| Sterile water | Add to 25 mL |

1.2.2 Exo III Digestion

Exo III digestion at 37° C. for 1 h and inactivated at 75° C. for 10 min. Scaling up accordingly when performing several reactions at the same time.

TABLE 8
Exo III digestion reaction

| COMPONENTS | 28 mL of REACTION |
|---|---|
| 10x NEBuffer 1 | 2.8 mL |
| Plasmid from last step | 25 mL |
| Exo III | Actual addition: 30000 |
| | U(300 μL, 100 U/μL) |

1.3 Purify clDNA with Gel Filtration Chromatography and Isopropanol

1.3.1 Gel Filtration Chromatography
Buffer A: 10 mM Tris-HCl, pH 7.5
Column: Bestarose 6 FF 153 mL
Sample: 28 ml
Flow: 60 cm/h
Collect fraction: 20 mAU-20 mAU, 40 mL
CIP: 1 M NaOH+pure water
Storage: pure water

1.3.2 Endotoxin Removing and Isopropanol Precipitation

Add 3M sodium acetate and 15% Triton-114 to the sample from last step and mix by vortexing shown as table 6. Keep the sample at 4° C. for 5 min. Then, centrifuge at 12000 g for 20 min at 25° C. After centrifugation, collect supernatant and add the equal volume of isopropanol to the supernatant and mix completely. Keep the sample at room temperature for 5 min. After that, centrifuge at 12000 g for 20 min and remove the supernatant. Finally, suspend the precipitate with 10 mM Tris-HCl (pH 7.5).

TABLE 9
Triton-114 system

| Material | Addition amount (A is the volume of cIDNA) |
|---|---|
| cIDNA | A(40 mL, ~100 μg/mL) |
| 15% Triton 114 | 0.1 A(4 mL) |

After three steps of enzyme digestion, gel chromatography, Triton 114 treatment and isopropanol precipitation, the eGFP_BSal_clDNA was made successfully. The DNA homogeneity (%) of the sample according to HPLC chromatogram was 97%. Endotoxin of the sample <10 EU/mg.

B. Obtaining oDNA (clDNA with Modified Nucleotides) from clDNA Via RCA

This experiment is designed to produce clDNA containing customized adaptors from the eGFP_BSal_clDNA obtained in the section above by Trueprime-RCA Kit (based on two enzymes: TthPrimPol, as DNA primase, and Phi29 DNA polymerase) and TeIN.

TABLE 10
Summary of experimental instruments

| Instrument | Brand/manufacturer | Model |
|---|---|---|
| Balance | Mettler Toledo | ME4002E |
| pH meter | INSEA | PHSJ-5 |
| Centrifuge | ThermoFisher | Heraeus ™ |
| | | Pico ™ 21 |
| Clean bench | AIRTECH | SW-CJ-2FD |

TABLE 11
Summary of material information

| No. | Material name | Brand or Manufacturer | Cat. No. |
|---|---|---|---|
| 1 | Exonuclease III | NEB | M0206 |
| 2 | NEBuffer 1 | NEB | M0206 |
| 3 | TritonX-114 | Solarbio | T8210 |
| 4 | Isopropyl alcohol | Sinopharm Chemical | 67-63-0 |
| | | Reagent Co., Ltd. | |
| 5 | KpnI | NEB | R3142L |
| 6 | HindIII | NEB | R3104S |
| 7 | CutSmart buffer | NEB | B7204S |
| 8 | 4BBTM TruePrime ® | 4basebio | 390100 |
| | RCA kit | | |
| 9 | Buffer D | 4basebio | 390100 |
| 10 | Buffer N | 4basebio | 390100 |
| 11 | Reaction Buffer | 4basebio | 390100 |
| 12 | Enzyme 1 | 4basebio | 390100 |
| | (TthPrimPol) | | |
| 13 | Enzyme 2 | 4basebio | 390100 |
| | (Phi29 DNA | | |
| | polymerase) | | |
| 14 | TelN | GenScript | NA |
| 15 | TelN buffer | GenScript | NA |
| 16 | AxyPrep DNA Gel | Axygen | AP-GX-250 |
| | Extraction Kit | | |
| 17 | Buffer DE-B | Axygen | AP-GX-250 |
| 18 | Buffer W1 | Axygen | AP-GX-250 |
| 19 | Buffer W2 | Axygen | AP-GX-250 |
| 20 | T4 ligase | NEB | M0202T |
| 21 | T4 ligase buffer | NEB | M0202T |
| 22 | BsaI | NEB | R3733L |
| 23 | T4 PNK | NEB | M0201L |
| 24 | T4 PNK buffer | NEB | M0201L |

### 1.1 RCA

Always mix by pipetting. DO NOT VORTEX
Transfer 10 μl of clDNA (≥1 ng/μl) into a clean tube
Add 10 μl of Buffer D and incubate at room temperature for 3 minutes
Neutralize the reaction by adding 10 μl of Buffer N to each tube
Keep the samples at room temperature until use*
Prepare the amplification mix adding the components in the order listed in the following table
Incubate at 30° C. for 3 hours**. Inactivate the reaction at 65° C. for 10 minutes.
Cool down to 4° C. Store amplified DNA at 4° C. for short-term storage or −20° C. for long-term storage.
(*) It is highly recommended to perform the amplification reaction just after the sample has been denatured.
(**) Incubation time can be increased up to 6 hours if higher amplification yields are required.

Scaling up accordingly when performing several reactions at the same time.

TABLE 12
RCA-100 ul system

| Material | Add amount | | Comments |
|---|---|---|---|
| cIDNA | 10 | uL | (>1 μg/mL, total 80 ng) |
| Buffer D | 10 | uL | 3 min at RT |
| Buffer N | 10 | uL | Neutralization |
| H₂O | 37.2 | μL | Amplification mix |
| Reaction buffer | 10 | μL | |
| dNTPs | 10 | μL | |
| Enzyme 1 | 10 | μL | |
| (TthPrimPol) | | | |
| Enzyme 2 (Phi29 | 2.8 | μL | |
| DNA polymerase) | | | |

1.2 Purify RCA Product (Concatamers) with Isopropanol (as Described Above)
1.3 Purify RCA Product (Concatamers) with Axygen Kits. (Optional)

If the sample is no more than 100 uL, Axygen kit could also be used to purify clDNA. The protocol is described below and bottles containing buffers labeles as described:
1) Add 2×sample volume of Buffer DE-B, mix.
2) Place a Miniprep column into a 2 ml microfuge tube. Transfer the sample from last step into the column. Centrifuge at 12,000×g for 1 minute.
3) Discard the filtrate from the 2 ml microfuge tube. Return the Miniprep column to the 2 ml microfuge tube and add 500 μl of Buffer W1. Centrifuge at 12,000×g for 30 seconds.
4) Discard the filtrate from the 2 ml microfuge tube. Return the Miniprep column to the 2 ml microfuge tube and add 700 μl of Buffer W2. Centrifuge at 12,000×g for 30 seconds
5) Discard the filtrate from the 2 ml microfuge tube. Place the Miniprep column back into the 2 ml microfuge tube. Add a second 700 μl aliquot of Buffer W2 and centrifuge at 12,000×g for 1 minute
6) Transfer the Miniprep column into a clean 1.5 ml microfuge tube (provided). To elute the DNA, add 50 μl of 10 mM Tris-HCL (pH 7.5) to the center of the membrane. Let it stand for 1 minute at room temperature. Centrifuge at 12,000 xg for 1 minute.

### 1.4 Oligo Denaturation and Annealing

Oligo (e.g. from table 3: oligo 21 or oligo 41) was denatured at 95° C. for 10 min and annealed naturally at room temperature for 30 min. Scaling up accordingly when performing several reactions at the same time.

TABLE 13
Oligo denaturation and annealing

| Material | Add amount |
|---|---|
| Phosphorylated Oligo | 95 μL |
| 20X SSC | 5 μL |

1.5 Oligo Phosphorylation (Optional, Skip this Step if the Oligo is Already Phosphorylated)

Oligo phosphorylation at 37° C. for 1 h.

TABLE 14
Oligo phosphorylation

| Material | Add amount |
|---|---|
| Oligo without phosphorylation | 80 μL |
| T4 PNK buffer | 10 μL |
| T4 PNK | 10 μL |

### 1.6 Bsal Digestion

Bsal digestion at 37° C. for 2 h and inactivated at 75° C. for 10 min.

TABLE 15
100 uL digestion system of BsaI

| Material | Add amount |
|---|---|
| purified RCA product | 85 μL (~100 ng/μL) |
| CutSmart buffer | 10 μL |
| BsaI | 20 U/μL, 1 μL |
| Sterile water | Add to 0.1 mL |

1.7 Purify Bsal-Digested RCA Product with Isopropanol (as Described Above)
1.8 Purify Bsal-Digested RCA Product with Axygen Kits. (Optional the Sample is No More than 100 uL; as Described Above)

### 1.9 T4 Ligation

T4 ligation at 16° C. overnight and inactivated at 75° C. for 10 min.

TABLE 16
100 uL T4 ligation system

| Material | Add amount |
|---|---|
| Oligo | 5 uL(~1 μg/μL) |
| BsaI-digested cIDNA | 85 uL (~100 ng/uL) |
| T4 ligase buffer | 10 μL |
| T4 ligase | 20000 U, 1 μL |
| Sterile water | Add to 0.1 mL |

### 1.10 Advanced Golden Gate Assembly (Optional)

Conventional ligation methods usually require several cloning steps to generate a construct of interest. At each step, a single DNA fragment is transferred from a donor plasmid or PCR product to a recipient vector.

While Golden Gate cloning, allows assembling up to fifteen fragments at a time in a recipient plasmid. Cloning is performed by pipetting in a single tube all plasmid donors, the recipient vector, a type IIS restriction enzyme and ligase, and incubating the mix in a thermal cycler. So we would also suggest to make oDNA with Golden Gate Assembly. The system and condition were described as table 14 and 15, respectively. Scaling up accordingly when performing several reactions at the same time

TABLE 17
Advanced Golden Gate Assembly system

| Material | Add amount | |
|---|---|---|
| 10X T4 ligase buffer | 10 | μL |
| cIDNA(amplified by RCA) | 24 | μg |
| Oligo | 72 | μg |

| | |
|---|---|
| BsaI | (100 U) 5 uL |
| T4 DNA ligase | (20000 U) 1 uL |
| Nuclease-free water | up to 200 μL |

TABLE 18
Advanced Golden Gate Assembly condition

| | |
|---|---|
| Temperature | Time |

| | | | |
|---|---|---|---|
| 37° | C. | 3 min | 25 cycles |
| 22° | C.* | 5 min | |

| | | |
|---|---|---|
| 22° | C. | 60 min |
| 50° | C. | 5 min |
| 80° | C. | 10 min |
| 4° | C. | Store the sample at 4° C. |
| | | until use |

*The optimal temperature of T4 ligase from NEB is 16° C. and 22° C. for T4 ligase from Thermofisher.

### 1.11 Digestion of Unexpected DNA

Exo III digestion at 37° C. for 1 h and inactivated at 75° C. for 10 min. Scaling up accordingly when performing several reactions at the same time.

TABLE 19
Exo III digestion reaction

| COMPONENTS | 0.3 mL of REACTION | |
|---|---|---|
| 10x NEBuffer 1 | 30 | μL |
| Plasmid from last step | 0.2 | mL |

| | |
|---|---|
| Exo III | 200 U(2 μL, 100 U/μL) |
| Nuclease-free water | up to 300 μL |

1.12 Purify oDNA with isopropanol (described above)
1.13 Purify oDNA with Axygen Kits. (Optional the Sample is No More than 100 uL; as Described Above)
The eGFP_BSal_oDNA was successfully made with oligos 21 and 41:

TABLE 20

| | Conc. | Volume | Total | Homogeneity |
|---|---|---|---|---|
| Sample | (ng/μL) | (ml) | (μg) | (%) |
| oDNA 21 | 119.6 | 0.05 | 6.0 | 95.6 |
| oDNA 41 | 130.0 | 0.05 | 6.5 | 96.2 |

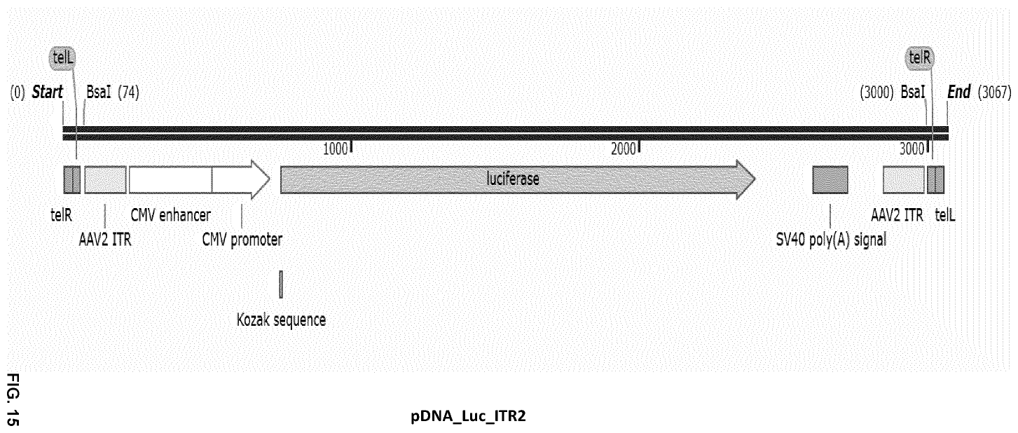

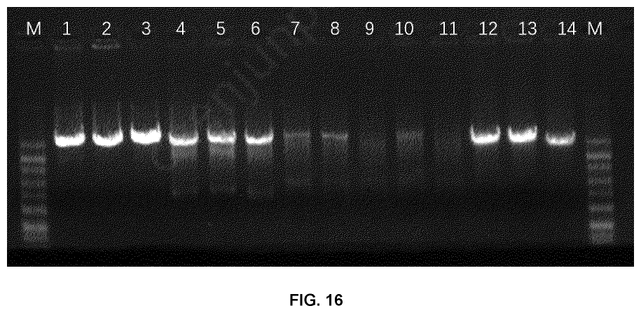

The same procedure was used to prepare clDNAs starting from Luc plasmid having SEQ ID NO: 17 (which comprises the sequence encoding for luciferase flanked by Bsal restriction sites, as well as protelomerase target sequences) and oligos 15, 37, 4, 28, 29, 17, 22. 37, 28, 29, 19 and 22 (see table 3). The same procedure was also used to prepare clDNAs starting from Luc-ITR plasmid having SEQ ID NO: 18 (wherein the sequence of interest additionally comprises ITRs flanking the sequence encoding for luciferase which is flanked by Bsal restriction enzyme as well as protelomerase target sequences, see FIG. 15) and oligo 37; thus, leading to oDNA 37_{ITR} (5.6 μg in total)—FIG. 16 (agarose gel electrophoresis).

The stability of the produced clDNAs was studied according to International Conference on Harmonization (ICH) over 36 months at −20° C. All the synthesized clDNAs including the modified nucleotides presented stability values suitable for use in gene therapy.

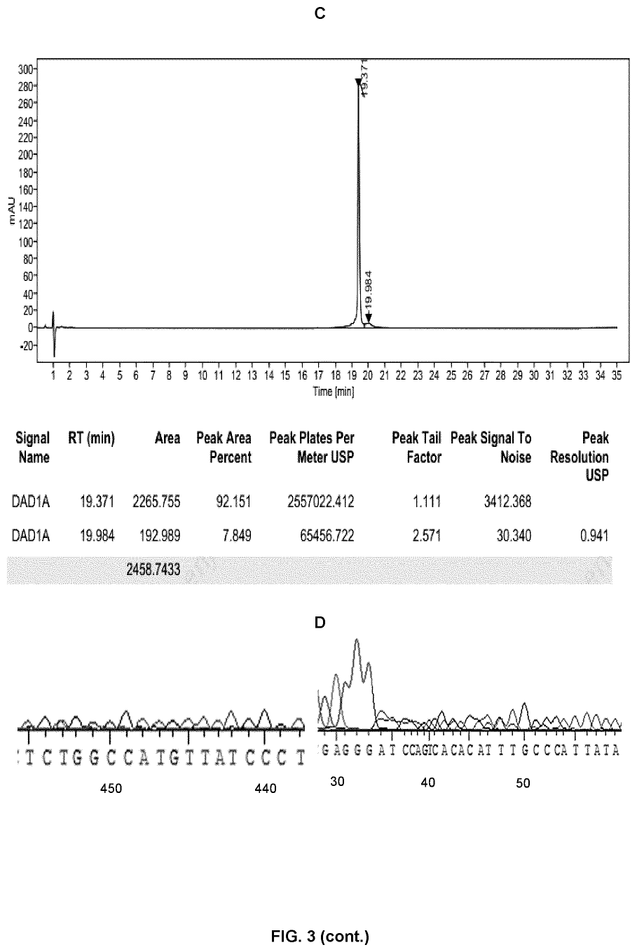

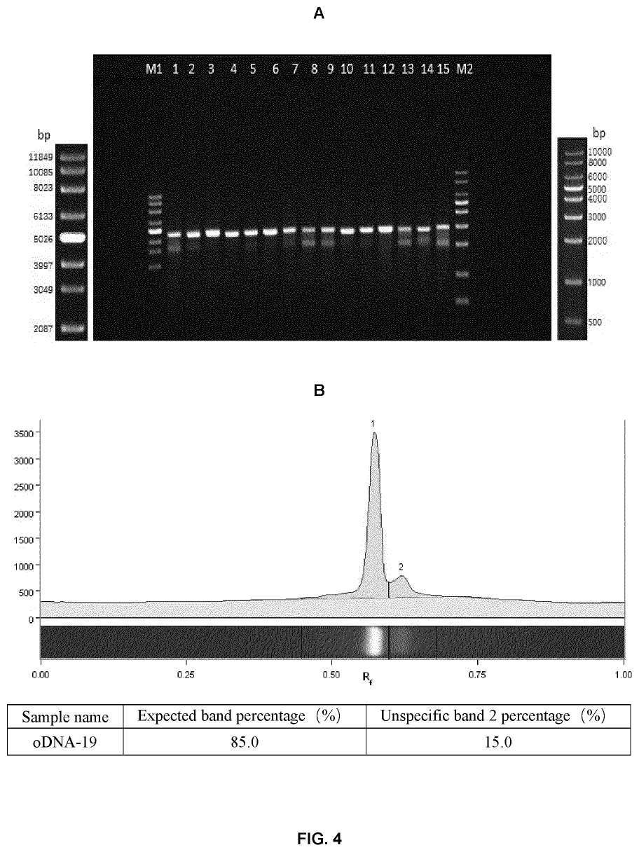

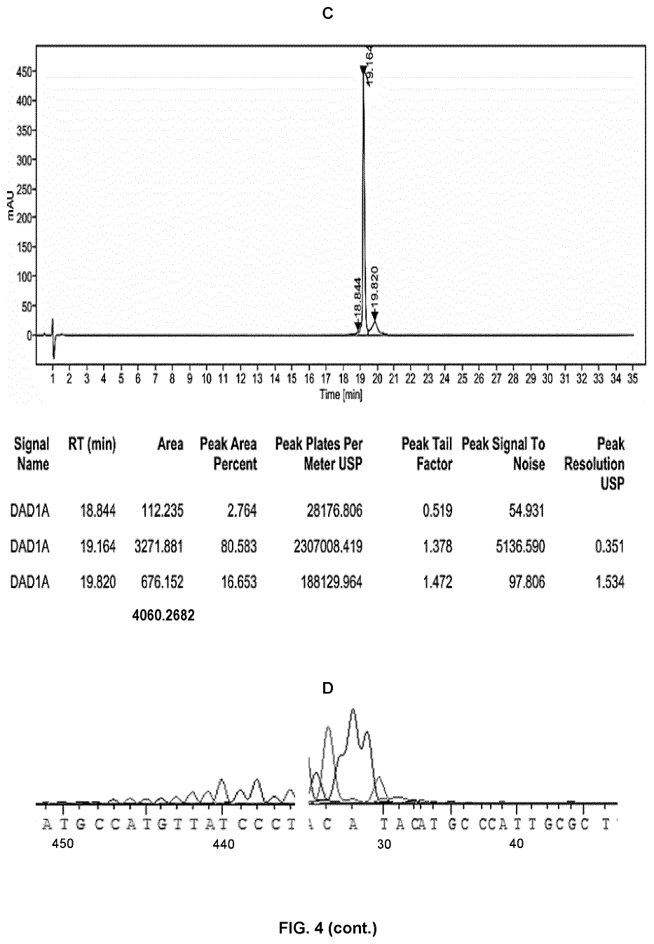

The quality of the obtained clDNA was determined by standard procedures, in particular, Agarose gel electrophoresis, Grayscale analysis, anion-exchange chromatography-HPLC and Sanger Sequencing. It was found that all clDNAs showed good quality features in terms of purity, peak resolution and sequence confirmation. For illustration, results for oDNA17, oDNA19 and oDNA41 quality control are shown in FIGS. 3, 4 and 5, respectively.

### Example 2. Functional Performance of clDNAs Containing at Least Two Modified Nucleotides

The clDNAs termed as oDNA 15, oDNA 37, oDNA 4, oDNA 28, oDNA29, oDNA17, oDNA22. oDNA 37, oDNA 28, oDNA 29, oDNA 19 and oDNA 22 obtained in example 1 were transfected on HaCaT cells and luciferase activity was determined.

Transfection. HaCaT cells (#EP-CL-0090, Elabscience. Batch #83000282208) were cultured using DMEM high glucose (Gibco #61965-059) containing 10% of Fetal Bovine Serum (Hyclone #SV30160.03H1). The day before of transfection cells were tripsinized and plated on 96-well plates (Greiner Bio-one #655090) at 6,000 cells/well in a final volume of 100 μl/well. Plates were placed at 5% C02 and 37° C. until the next day. Just before transfection the medium was removed by aspiration and 90 μl/well were added. Transfection was carried out at 100 ng DNA/well using PEI (jetPEI® Polyplus #101-10N) or CXP037 (see example 4 below) as vehicle for transfection at N/P ratios of 5 and 30, respectively. The transfection mixtures were prepared using DPBS (Hyclone, #SH30028.02, Thermo Fisher) following instructions from jetPEI manufacturer. More specifically, 100 ng of DNA were complexed with jetPEI at N/P ratio of 5 being N/P the number of nitrogen residues (N) in jetPEI per phosphate (P) in DNA, following the formula: N/P ratio=7.5*×ul of jetPEI/3×μg of DNA. In the case of transfection with CXP037, a NP ratio of 30 was used (15.8 ug of CXP037/ug of DNA).

Luciferase activity. At the indicated incubation times, luciferase activity was determined by adding 100 μl/well of commercial reagent BrightGlo (Promega #E2620) directly to the wells. After 5 minutes of incubation at room temperature in the dark, luminescence was quantified using the VictorNivo (PerkinElmer) plate reader. Luminescence of the individual wells were normalized using a control pDNA(Luc) at day 1.

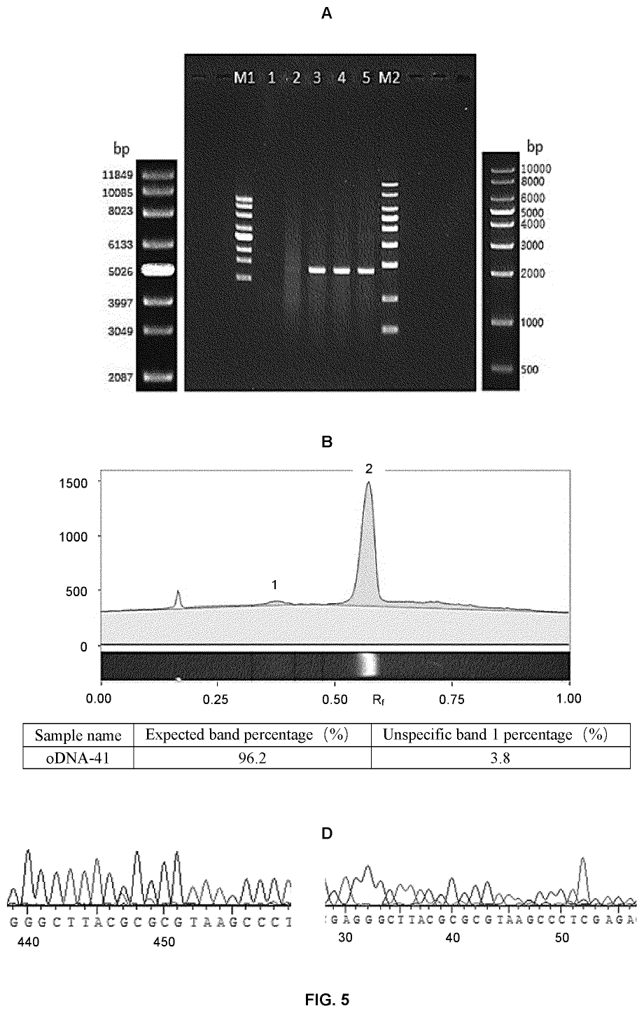

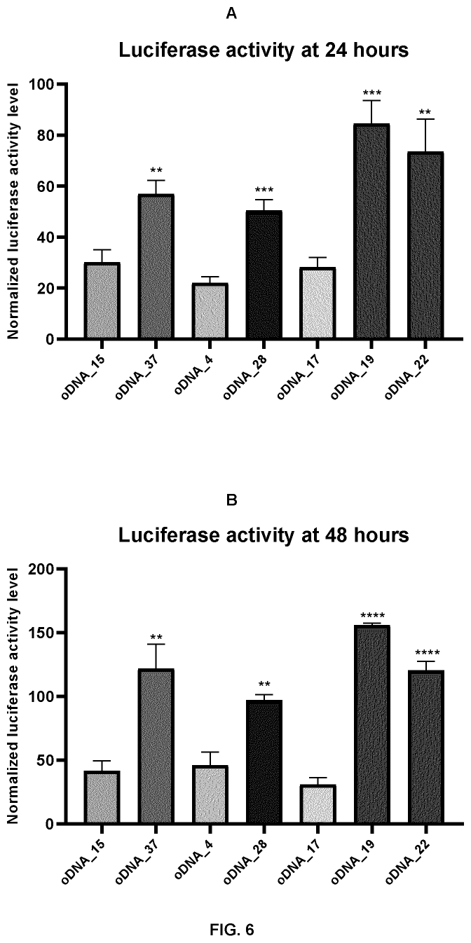

Results are shown in FIGS. 6 and 7. FIG. 6 shows that cells transfected with clDNAs containing at least two modified nucleotides showed significantly higher luciferase activity when compared to the corresponding clDNA with the natural nucleotides. This demonstrates that functional performance of the sequence of interest, in this case, luciferase, is much higher (statistically significant) when transfected within a clDNA containing at least two modified nucleotides. FIG. 7 shows the evolution of luciferase activity level vs time for the assayed clDNAs. It was observed that only those clDNAs containing modified nucleotides achieved a statistically significant increase in the level of luciferase activity at 48 h versus 24 hours.

### Example 3. Release of clDNA, Containing at Least Two Modified Nucleotides, from Polyplexes

This assay is based on the fluorescence of the picogreen fluorophore produced when this molecule binds to the free double strand DNA. Formed polyplexes were diluted 10 times in PBS and 10 μl/well were added to a 384-well plate in a final volume of 40 μl/well. A Heparin concentration of 8 U/ml, physiological conditions, was added to each diluted polyplex. After addition of picogreen, the fluorescence signal was taken after 12 hours. The fluorescence signals were converted to amount of DNA using a standard DNA curve contained in the plate. The assay permits to determine the amount of released DNA defined as the difference of DNA concentration in the absence and in the presence of the maximal heparin concentration (8 U/ml).

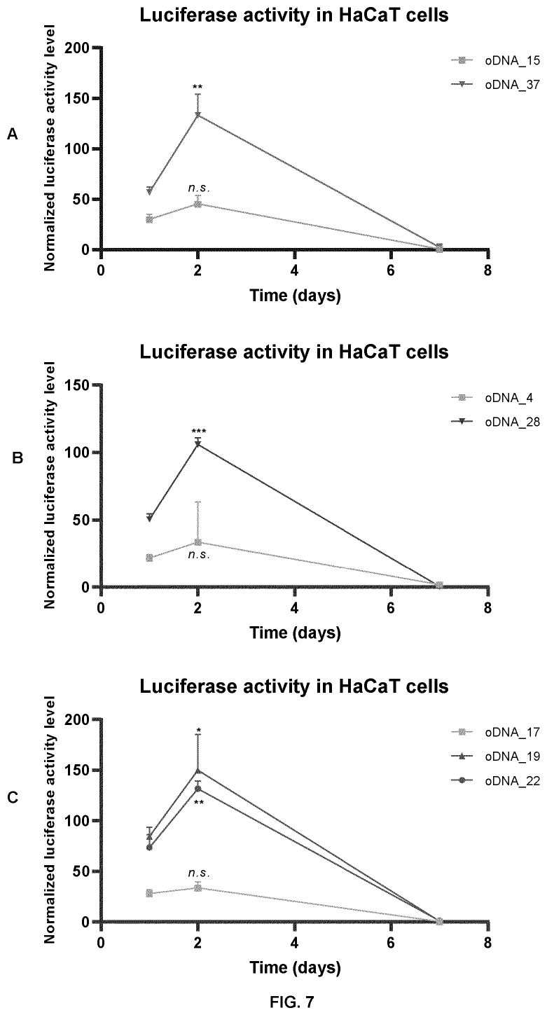

Results are shown in FIG. 8. Polyplexes formed by polymer CXP037 and different oDNAs, bearing natural and modified nucleotides, were monitored at physiological conditions (incubated with 8 U/mL of heparin) (Engelberg et al, 1961) for 12 hours, and DNA release quantified. Depending on the DNA utilized as cargo, different behaviour is observed; thus, polyplexes containing as cargo DNA bearing modified nucleotides show a statistically significant impact on the amount of “released DNA” yielding greater DNA availability than polyplexes with natural DNA.

Then, according to results from 12 hours incubation of these polyplexes in a physiological environment, DNA bioavailability is higher using these oDNA with modified nucleotides.

### Example 4. Synthesis and Characterization of Polymer CXP037

CXP037 is a polycationic polymer vehicle which forms polyplex micelles with the clDNAs for cell transfection.

General considerations: Reactions were carried out under a nitrogen atmosphere unless otherwise stated. Solvents, including NMP (1-Methyl-2-pyrrolidinone >99%), anhydrous CH₂Cl₂ and anhydrous DMF, were purchased from Aldrich and used as disposed. All reagents were obtained from commercial suppliers and used without further purification. The polymerization reaction was monitored with IR (CARY 630 ATR-FTIR SPECTOMETER). The aminolysis reaction was purified by centrifugal-assisted ultrafiltration using viva-spin 3000 MWCO PES.

NMR spectroscopy: ¹H spectra were recorded on a 300 MHz Bruker Advance AC-300 spectrometer.

SEC-MALS: Size-exclusion chromatography coupled to a multi-angle light-scattering photometer (SEC-MALS) measurements were performed using MALVERN GPC MAX with detector TDA MALVERN 305 equipped with UV—RI-RALS-MALS. The separation were carried out at room temperature using successively cationic column TSKgel G3000PWXL-CP with a precolumn in 0.1 M solution of NaNO₃ with 0.005% NaN₃ at a flow rate of 1 mL min⁻¹. The masses of the samples injected onto the column were typically 2-5 mg, whereas the solution concentration was 10-20 mg mL⁻¹. For the data acquisition and evaluation OMNISEC 5.12 software.

pKa determination procedure: The pKa of a cationic polymer is determined by acid-base titration, measuring the pH of the solution throughout the process. The pKa is then obtained from the titration graph. To carry out the measurement, 1 mg/mL solution of the cationic polymer is prepared in Milli-Q water and a known quantity of HCl 0.1M is added until the pH of the solution is around 2. At this point, the titration is performed with NaOH 0.2M using an automatic Methrom 916 titouch potentiometer with a Dosino 800 dispenser. The titration speed is set to 0.1 mL/min with a signal drift of 50 mV/min. The titration is complete when the pH reaches 12. The instrument measures the pKa of the chemical species present and generates a .txt report. If the instrument identifies many equivalence points that don't correspond with the chemical nature of the compound, the pKa is determined manually using graphical methods.

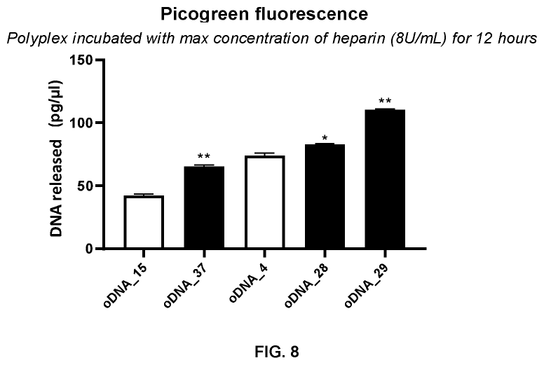

### Synthetic Route of PAspDET/DIIPA-Compound CXP037A. Shown in FIG. 9A

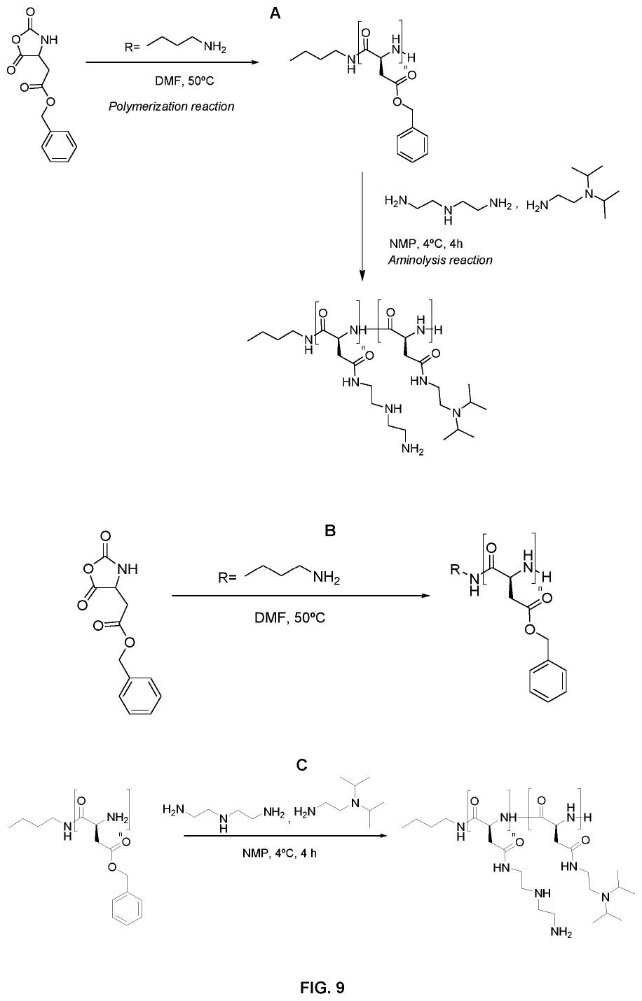

Synthesis of poly(β-benzyl L-aspartate) (PBLA). Shown in FIG. 9B. PBLA was synthesized following the general procedure for the ring-opening polymerization of the NCA, using n-butylamine as the initiator. The polymerization reaction was carried out in a flame-dried Schlenk flask under a nitrogen atmosphere. First, the BLA NCA (3 g, 12 mmol) was dissolved in a mixture of dry dichloromethane (120 mL) and DMF (10 mL). Then, a solution of the initiator (n-butylamine, 11.89 μL, 0.12 mmol) in DMF (2 mL) was added to the reaction mixture. The mixture was stirred at 50° C. for 16 hours. Upon completion, the reaction mixture became clear and full conversion of the monomer could be detected by IR. The reaction mixture was poured into diethyl ether to precipitate the product. The precipitate was isolated by centrifugation (3750 rpm, 4 min) and dried under vacuum. PBLA was isolated as a white solid (1.5 g, η=60%). The 1H NMR spectrum of PBLA is shown in FIG. 10.

### Synthesis of PAsp(DET/DIIPA)-Compound CXP037A. Shown in FIG. 9C

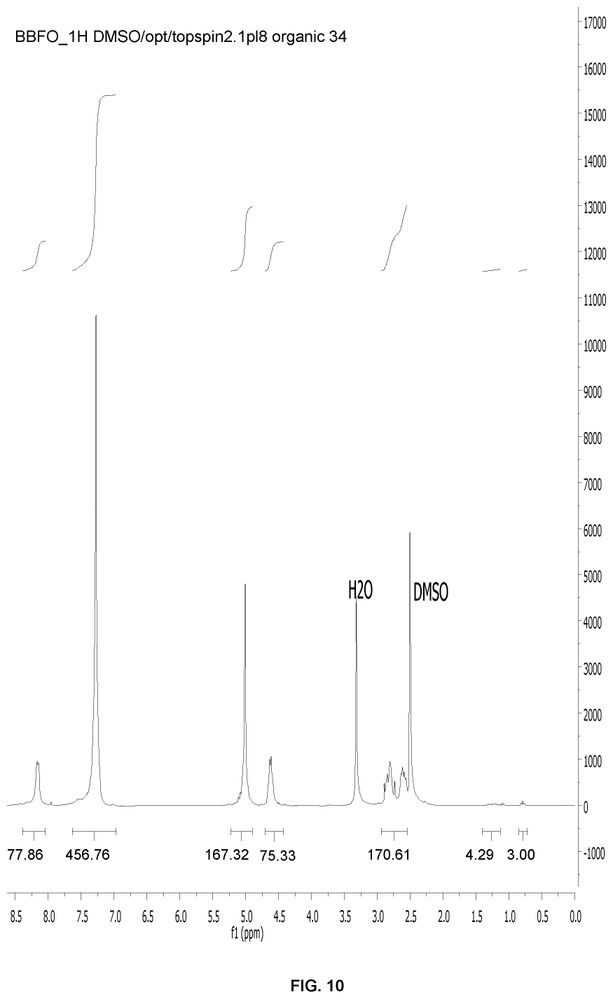

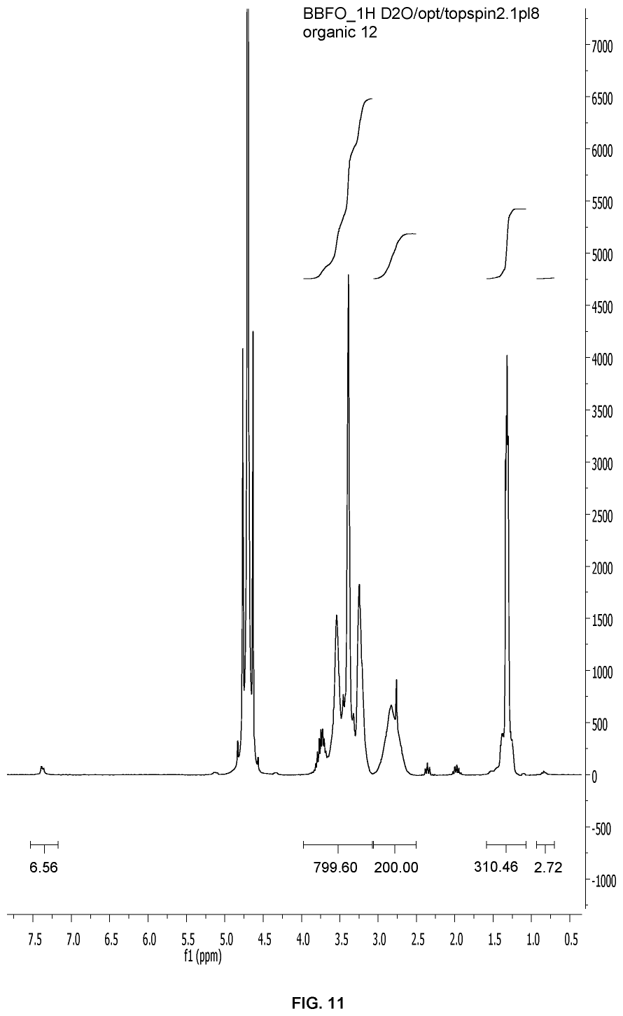

PAsp(DET/DIIPA) was prepared by an aminolysis reaction over PBLA with DET and DIIPA. PBLA (DP=67, 60 mg) was dissolved in NMP (3 mL) and cooled to 4° C. This solution was added dropwise to the mixture solution of DET (50 eq DET vs unit of Asp, 1.58 mL) and DIIPA (100 eq DIIPA vs unit of Aspartic, 5.18 mL), cooled at 4° C. and the mixture was stirred for 4 hours at the same temperature. After this time, the reaction mixture was added dropwise into cold HCl 6M for neutralization (pH: 3.5). The polymer product was purified by centrifugal-assisted ultrafiltration. After filtration, the remaining aqueous polymeric solution was lyophilized to obtain the final product (42 mg, η=64%). FIG. 12 shows SEC-MALS-RI of CXP037A Analysis for MW determination. It may be observed that MW=14000 Da (1.03). FIG. 11 shows 1H NMR spectrum of CXP037.

### CITATION LIST

Kapp K et al., “EnanDIM—a novel family of L-nucleotide-protected TLR9 agonists for cancer immunotherapy” 2019, J Immunother Cancer., vol 7(1), pp. 5
Heinrich J. et al., “Linear closed mini DNA generated by the prokaryotic cleaving-joining enzyme TeIN is functional in mammalian cells” 2002, J Mol Med, vol. 80(10), pp. 648-54
Xiao X. et al., “A novel 165-base-pair terminal repeat sequence is the sole cis requirement for the adeno-associated virus life cycle”, 1997, J Virol., vol. 71(2), pp. 941-948.
Altschul et al., “Basic local alignment search tool”, 1990, J. Mol. Biol, vol. 215, pp. 403-410.
WO2011000997
U.S. Pat. No. 4,373,071
EP1859812
Engelberg H. Plasma heparin levels in normal man. Circulation. 1961; 23:578-581. doi: 10.1161/01.CIR.23.4.578.
Beaucage S. L. et al, Deoxynucleoside phosphoramidites-A new class of key intermediates for deoxypolynucleotide synthesis. Tetrahedron Letters, Volume 22, Issue 20, 1981, Pages 1859-1862

1. A closed linear DNA (clDNA) consisting of a stem region comprising a double stranded DNA sequence of interest covalently closed at both ends by hairpin loops, the clDNA comprising at least two modified nucleotides.

2. The clDNA according to claim 1, wherein:
the at least two modified nucleotides are located in one or both single stranded end loops of the clDNA;
at least one modified nucleotide is located in one of the single stranded end loops and at least another modified nucleotide is located in one of the strands forming the stem region of the adaptors of the clDNA; or, alternatively,
the at least two modified nucleotides are in one or both strands forming the stem region of the adaptors of the clDNA.

3. The clDNA according to claim 2, wherein when the at least one modified nucleotide is in one of the strands forming the stem region, the modified nucleotide is located:
within the strand region defined by the nucleotides at positions 1 to 5 with respect the last nucleotide forming the loop; or, alternatively,
within the strand region defined by the nucleotides 1 to 10 with respect to the last nucleotide forming part of the DNA sequence of interest.

4. The clDNA according to claim 1, wherein the at least two modified nucleotides are independently selected form the group consisting of 2-amino-deoxyadenosine, 5-methyl-deoxycytidine, thiophosphate nucleotide, inosine nucleotide, locked nucleic acid (LNA) nucleotide, L-DNA nucleotide, 8-oxo-deoxyadenosine nucleotide, and 5-Fluoro-deoxyuracil nucleotide.

5. The clDNA according to claim 1, which comprises from 3 to 20 modified nucleotides, for example, 4, 5, 6, 7, 8, 9, 10, 11, 12, 13, 14, 15, 16, 17, 18, 19 or 20 modified nucleotides.

6. The clDNA according to claim 1 which comprises at least two LNA nucleotides.

7. The clDNA according to claim 1 which comprises two LNA nucleotides.

8. The clDNA according to claim 1 which comprises at least two thiophosphate nucleotides.

9. The clDNA according to claim 1, wherein the stem region comprises two restriction sites flanking the DNA sequence of interest.

10. The clDNA according to claim 1 wherein the clDNA comprises a primase recognition site.

11. The clDNA according to claim 1, wherein the at least one of the loops comprises a primase recognition site.

12. The clDNA according to claim 1, wherein the sequence of interest comprises inverted terminal repeats (ITR).

13. The clDNA according to claim 1, wherein the DNA sequence of interest comprises an expression cassette.

14. (canceled)

15. A pharmaceutical composition comprising a therapeutically effective amount of the clDNA according to claim 1 and pharmaceutically acceptable carriers or excipients.

16.-22. (canceled)

23. A composition comprising a carrier and the clDNA according to claim 1.

24. The composition according to claim 23, wherein the carrier is a gene vector.

25. The composition according to claim 24, wherein the gene vector is a viral vector.

26. The composition according to claim 25, wherein the gene vector is a non-viral vector.

27. The composition according to claim 26, wherein the non-viral vector is a polycationic polymer.

28. (canceled)

29. (canceled)
